(19) United States
(12) Patent Application Publication
(10) Pub. No.: US 2022/0214301 A1
(43) Pub. Date: Jul. 7, 2022

(54) METHODS AND APPARATUS FOR ERROR MITIGATION AND DIFFERENCE DETERMINATION

(71) Applicant: GlySens Incorporated, San Diego, CA (US)

(72) Inventors: Piyush Gupta, San Diego, CA (US); Krista Bertsch, Poway, CA (US)

(21) Appl. No.: 17/569,274

(22) Filed: Jan. 5, 2022

Related U.S. Application Data
(60) Provisional application No. 63/134,869, filed on Jan. 7, 2021.

(51) Int. Cl. G01N 27/327; G01N 33/49; A61B 5/145; A61B 5/1486

(52) U.S. Cl. CPC G01N 27/3273; G01N 27/3274; A61B 5/14865; A61B 5/14532; G01N 33/4925

(57) ABSTRACT

Apparatus and methods for mitigation of calibration error and determination of mean absolute relative difference (MARD) values in for example a blood analyte sensor or system. In one exemplary embodiment, the apparatus and methods include i) intelligently collecting reference data in order to enhance a calibration calculation, ii) identification and compensation of systematic error based on spatial heterogeneity between sensors of a differential sensor pair, and/or iii) identification/selection of portions of available calibration points that provide calibration curves with the best MARD values. The apparatus and methods can provide more accurate blood analyte sensor calculations and improved user experience.

### PRIORITY AND RELATED APPLICATIONS

This application claims priority benefit of U.S. Provisional Patent Application Ser. No. 63/134,869 filed Jan. 7, 2021 and entitled “Methods and Apparatus for Error Mitigation and Difference Determination,” which is incorporated herein by reference in its entirety.

This application is generally related to portions of the subject matter of co-owned and co-pending U.S. patent application Ser. No. 13/559,475 filed Jul. 26, 2012 entitled “Tissue Implantable Sensor With Hermetically Sealed Housing,” Ser. No. 14/982,346 filed Dec. 29, 2015 and entitled “Implantable Sensor Apparatus and Methods”, Ser. No. 15/170,571 filed Jun. 1, 2016 and entitled “Biocompatible Implantable Sensor Apparatus and Methods”, Ser. No. 15/197,104 filed Jun. 29, 2016 and entitled “Bio-adaptable Implantable Sensor Apparatus and Methods”, Ser. No. 15/359,406 filed Nov. 22, 2016 and entitled “Heterogeneous Analyte Sensor Apparatus and Methods”, Ser. No. 15/368,436 filed Dec. 2, 2016 and entitled “Analyte Sensor Receiver Apparatus and Methods”, and Ser. No. 15/472,091 filed Mar. 28, 2017 and entitled “Analyte Sensor User Interface Apparatus and Methods,” each of the foregoing incorporated herein by reference in its entirety.

This application is also generally related to portions of the subject matter of co-owned and co-pending U.S. patent application Ser. No. 15/645,913 filed Jul. 10, 2017 entitled “Analyte Sensor Data Evaluation and Error Reduction Apparatus and Methods” and U.S. patent application Ser. No. 16/233,536 filed Dec. 27, 2018 entitled “Apparatus and Methods for Analyte Sensor Mismatch Correction,” each of the foregoing incorporated herein by reference in its entirety.

This application is also generally related to portions of the subject matter of co-owned and co-pending U.S. patent application Ser. No. 16/443,684 filed Jun. 17, 2019 and entitled “Analyte Sensor Apparatus and Methods,” which claims the benefit of priority to co-owned and co-pending U.S. Provisional Patent Application No. 62/687,115 filed on Jun. 19, 2018 and entitled “Analyte Sensor Apparatus and Methods,” as well as co-owned and co-pending U.S. patent application Ser. No. 16/453,794 filed Jun. 26, 2019 and entitled “Apparatus and Methods for Analyte Sensor Spatial Mismatch Mitigation and Correction,” which claims the benefit of priority to co-owned and co-pending U.S. Provisional Patent Application No. 62/690,745 filed on Jun. 27, 2018 and entitled “Apparatus and Methods for Analyte Sensor Spatial Mismatch Correction,” each of the foregoing incorporated herein by reference in its entirety

This application is also generally related to portions of the subject matter of co-owned and co-pending U.S. Provisional Patent Application Ser. No. 63/179,910 filed Apr. 26, 2021 and entitled “Methods and Apparatus for Substance Delivery in an Implantable Device,” which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### 1. TECHNICAL FIELD

The disclosure relates generally to the field of data analysis and processing, including for e.g., sensors, therapy devices, implants and other devices (such as those which can be used consistent with human beings or other living entities for in vivo detection and measurement or delivery of various solutes), and in one exemplary aspect to methods and apparatus enabling the use of such sensors and/or electronic devices for, e.g. monitoring of one or more physiological parameters, including through use of error identification, analysis, and/or correction routines or computer programs to enhance the accuracy and reliability of such physiological parameter measurements.

### 2. DESCRIPTION OF RELATED TECHNOLOGY

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use within a living subject (i.e., in vivo) can now be surgically implanted within such subjects with minimal effect on the recipient subject, and in fact convey many inherent benefits.

One particular area of note relates to blood analyte monitoring for subjects, such as for example glucose monitoring for those with so-called “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of both of these phenomena. Safe and effective correction of this dysregulation requires blood glucose monitoring.

Currently, glucose monitoring in the diabetic population is based largely on collecting blood by “fingersticking” and determining its glucose concentration by conventional assay. This procedure has several disadvantages, including: (1) the discomfort associated with the procedure, which should be performed repeatedly each day; (2) the near impossibility of sufficiently frequent sampling (some blood glucose excursions require sampling every 20 minutes, or more frequently, to accurately treat); and (3) the requirement that the user initiate blood collection, which precludes warning strategies that rely on automatic early detection. Using the extant fingersticking procedure, the frequent sampling regimen that would be most medically beneficial cannot be realistically expected of even the most committed patients, and automatic sampling, which would be especially useful during periods of sleep, is not available.

Implantable glucose sensors (e.g., continuous glucose monitoring sensors) have long been considered as an alternative to intermittent monitoring of blood glucose levels by the fingerstick method of sample collection. These devices may be fully implanted, where all components of the system reside within the body and there are no through-the-skin (i.e. percutaneous) elements, or they may be partially implanted, where certain components reside within the body but are physically connected to additional components external to the body via one or more percutaneous elements. Further, such devices (especially fully implantable devices) provide users a great deal of freedom from potentially painful (and not always optimally timed) intermittent sampling methods such as “fingersticking,” as well as having to remember and obtain self-administered blood analyte readings.

The accuracy of blood analyte detection and measurement is an important consideration for implanted analyte sensors, especially in the exemplary context of current blood glucose monitoring systems (such as e.g., fully implanted blood glucose sensor systems), and even the future development of implantable blood analyte monitoring systems, e.g., in support of the development of an artificial pancreas for the glucose analyte. Hence, ensuring accurate measurement for extended periods of time (and minimizing the need for any other confirmatory or similar analyses) is of great significance.

In conventional sensors, accuracy can be adversely affected by a myriad of factors such as e.g., random noise, foreign body response (FBR), other tissue responses, anoxia or hypoxia in the region of the analyte sensor, blood analyte tissue dynamics, an insufficient degree of vascularization in a given area being sensed, mechanical jarring, and/or other variables. Additionally, within architectures which use differential sensing (such as e.g., glucose/oxygen differential systems), the difference in position within the sensor of the glucose and reference oxygen electrodes may impart some inherent error; simply stated, the two electrodes/sets can only get so physically close to one another due to mechanical and chemical limitations, and hence error due to such difference may always be present in blood glucose signals generated by such sensors.

Useful data for calibration of a blood glucose measurement device may also arise from varying types of sources. For instance, in addition to the fingerstick method described above, other devices such as percutaneous or other “continuous” glucose monitoring systems may generate data which, if utilized properly, can provide insight into the calibration of other devices such as a fully implantable glucose monitoring device.

Accordingly, there is a need for: i) general improvement of sensor and blood analyte measurement/calibration accuracy, ii) methods and apparatus for in vivo determination and reduction of context-specific errors dues to unmodeled system variables, and iii) reduction and/or elimination of fingerstick testing during analyte sensor calibration.

Improved apparatus and methods addressing the foregoing needs would, in certain contexts, also ideally be able to assimilate data generated from other types of systems (such as percutaneous devices) to assist in calibration.

### SUMMARY

The present disclosure satisfies the foregoing needs by providing, inter alia, improved apparatus (including an implanted sensor and associated logic) and methods, for accurately providing information relating to sensed analyte levels and improving user experience.

In some embodiments, the disclosed improved apparatus and methods enable: (i) identifying time periods for obtaining reference data that are optimal for sensor calibration, (ii) using a secondary or ancillary continuous analyte monitoring device to obtain calibration reference data for a primary continuous analyte monitoring device, (iii) identifying and correcting for systematic error related to differential sensor signals, and/or (iv) provide for sensor calibration using an optimized hybrid linear regression and bootstrapping technique.

In a first aspect, an apparatus for use with an implantable blood analyte sensor apparatus is disclosed. In one embodiment, the apparatus includes data processing apparatus configured for data communication with an analyte sensor apparatus; and a storage apparatus in data communication with the processing apparatus. In one variant, the storage apparatus comprises a computer program which, when executed, causes the data processing apparatus to: (i) cause operation of the blood analyte sensor apparatus in an initial calibration mode; (ii) identify at least one time or time interval in which a reference analyte measurement should be taken for optimal calibration; i.e., where it would be most useful in calibration; (iii) obtain reference analyte measurement data during the at least one time or time interval; (iv) obtain a blood analyte measurement from the blood analyte sensor at same time (or as close thereto as possible) as the reference analyte measurement; and (v) use the reference analyte measurement, in conjunction with other reference measurements, to calibrate the implantable blood analyte sensor.

In one implementation, the computerized logic of the apparatus is configured such that steps (ii) through (iv) are repeated until a threshold number of data points are collected. In another implementation, steps (ii) through (iv) are repeated until a threshold accuracy or desired attribute of a calibration curve is reached. In yet another implementation, steps (ii) through (iv) are repeated for a predetermined duration of time.

In one configuration of the apparatus, the obtaining the reference analyte measurement and obtaining the blood analyte measurement includes: (i) receipt of time-stamped blood analyte reference data; and (ii) collection of time-stamped calculated blood analyte sensor data.

In one embodiment, the identifying the at least one optimal time interval includes identifying a time when the sensor is operating in a range where calibration points are needed. In one variant, the identifying is based on a calibration algorithm. In one implementation, the identifying is based on a glucose to oxygen ratio.

In one variant, the logic of the apparatus is configured to obtain the reference analyte measurement via at least: causing a notification to a user of the apparatus that a reference measurement should be obtained; and receiving the reference analyte measurement from the user (e.g., manually inputted by the user or transmitted from a fingerstick or other calibration device).

In another variant, the logic is configured to obtain the reference analyte measurement via at least: notifying a secondary or ancillary continuous analyte sensor (e.g., percutaneous CGM worn by the user) that a reference needs to be obtained, and causing the secondary continuous analyte sensor to take a reference measurement during the optimal time interval and communicate the reference measurement to the apparatus. In one configuration, the identification of the at least one optimal time interval allows for a more accurate calibration using the same number (or fewer) reference measurements as compared to a calibration not using identified time intervals. In some configurations, the identification of the at least one optimal time interval allows the apparatus to obtain an accurate calibration using fewer reference measurements. In varying implementations, the apparatus is disposed (i) on a fully implanted/implantable sensor apparatus and integrated therewith or (ii) on a receiver apparatus disposed external to a user within which the sensor apparatus is implanted.

In one variant of the apparatus, the implanted analyte sensor includes a glucose sensor (part of a so-called “continuous glucose monitor” or CGM), and the blood analyte measurement includes blood glucose concentration data. In one implementation, the glucose sensor is an oxygen-based glucose sensor. In another implementation, the glucose sensor includes a hydrogen peroxide-based glucose sensor (whether alone, or in tandem with an oxygen based sensor). In yet another implementation, the glucose sensor includes both a hydrogen peroxide-based sensor and oxygen-based glucose sensor which are logically communicative with one another in at least one aspect.

In one embodiment, the aforementioned apparatus is integral with the blood analyte sensor. In another embodiment, the apparatus is located at an external apparatus in communication with the blood analyte sensor. For instance, the external apparatus may be embodied as a dedicated receiver for the blood analyte sensor. In another implementation, the external apparatus is rendered as an application located on a user device (e.g., cell phone, laptop, wearable technology device), and/or in a cloud server.

In another aspect of the disclosure, an implantable blood analyte sensor apparatus is described. In one embodiment, the blood analyte sensor apparatus includes multiple sets of sensor elements, each set including at least an analyte sensor and a background analyte sensor, wherein the blood analyte sensor provides a differential analyte signal based on one or more analyte signals and one or more background analyte signals. In one configuration, the set of sensor elements is rendered as one or more pairs or groupings of sensor elements. In one implementation, the blood analyte sensor is an oxygen-based glucose sensor, and each pair includes a glucose sensor and one or more (background) oxygen sensors. In another implementation, each pair includes a glucose sensor and a (background) hydrogen peroxide sensor. In yet another implementation, each pair/set includes a glucose sensor and at least one of an oxygen or hydrogen peroxide sensor.

In one variant, the apparatus includes a storage apparatus having a computer program which, when executed, causes a data processing apparatus to: identify and compensate for systematic error in the blood analyte sensor. In one embodiment, the computer program causes the data processing apparatus to determine a true/ideal background analyte concentration present at a single analyte sensor using external reference data; calculate contributions from the plurality of background sensing elements to the true/ideal background analyte concentration at the single analyte sensor; and to use the calculated contributions to estimate the true/ideal background analyte concentration at the single analyte sensor during later analyte sensor operation (e.g., when no external reference data is available). In one implementation, the external reference data is reference data obtained from an external source such as a fingerstick test or a second blood analyte sensor that is worn or partially implanted on the user.

In another variant, the storage apparatus comprises a computer program which, when executed, causes the data processing apparatus to: use a hybrid (e.g., least squares and bootstrapping) calibration approach to calibrate the blood analyte sensor using a given set of calibration data points. In one embodiment, the computer program causes the data processing apparatus to calculate a plurality of calibration curves and identify the calibration curve of the plurality of calibration curves that provides the least mean absolute relative difference (MARD) between the calibration curve and external reference data.

In another aspect, a method of calibrating an implanted blood analyte sensor is disclosed. The method includes utilizing “intelligently” identified or collected calibration points to perform a calibration calculation. In one embodiment, the utilizing intelligently collected calibration points includes: (i) identifying time intervals when the sensor is operating in a desired range and/or under the user is within a desired physiologic state, and (ii) obtaining blood analyte sensor data and external analyte reference data during the time intervals.

In one aspect, a method of collecting reference/external analyte data that can be used to calibrate an implantable blood analyte sensor is disclosed. In one embodiment, obtaining external analyte reference data includes providing a notification to a user of the blood analyte sensor. In another embodiment, obtaining external analyte reference data includes instructing a second blood analyte sensor to take/provide a reference measurement. In one implementation, the second blood analyte sensor is a continuous and/or automatic blood analyte sensor (e.g., a continuous glucose monitor) that is temporarily worn and/or partially implanted on the user for the duration of calibration.

In one aspect, a method of identifying and compensating for systematic error in a blood analyte sensor is disclosed. In one embodiment, the blood analyte sensor includes multiple sets or pairs of sensor elements that each provide a differential sensor signal. In one variant, the systematic error is based on a difference in background/secondary analyte levels between differential element sets or pairs; e.g., oxygen-based glucose sensor and (background/secondary) oxygen sensor elements. In one embodiment, the method includes calculating a background analyte concentration for a first sensor element set or pair using background analyte detectors of at least one other sensor element set or pair. In one implementation, using the at least one other sensor element set or pair includes using all other sensor element sets or pairs of the blood analyte sensor, and calculating weights/contributions from a plurality of background sensors (from a plurality of sensor element sets or pairs) to a true/ideal background analyte concentration at a single sensor element (in one sensor element set/pair). In one implementation, the method includes performing a linear regression on contributions from every analyte sensor set or pair. In one embodiment, the method includes calibrating the blood analyte sensor after the identifying and compensating for the systematic error.

In another aspect, a method of operating an implanted blood analyte sensor is disclosed. In one embodiment, the implanted blood analyte sensor is subject to one or more sources of systematic error, and the method includes: obtaining first blood analyte data using the sensor, the obtained data subject to the one or more sources of error; obtaining reference data not subject to the one or more sources of error; evaluating the obtained blood analyte data and the reference data using one or more algorithms; generating an operational error correction model based at least on the evaluating; and applying the generated model to second blood analyte data to correct for at least one of the one or more sources of error. In one implementation, the evaluating includes calculating weights/contributions from a plurality of background sensors (e.g., oxygen sensors) to a true/ideal background analyte concentration at a single analyte sensor element (e.g., glucose sensor). In one embodiment, the generating the operational error correction model includes using the calculated weights to estimate the true background analyte concentration at a single analyte sensor element. In one implementation, the background analyte includes oxygen and the analyte includes glucose. In another implementation, the background analyte includes hydrogen peroxide and the analyte includes glucose. In another implementation, the analyte includes glucose and the background analyte includes oxygen and hydrogen peroxide.

In one variant, the method advantageously does not require identification or human understanding of one or more physical or physiologic mechanisms causing the at least one of the one or more sources of error.

In yet another aspect, a method of using bootstrapping-based calculations to calibrate a blood analyte sensor using a given set of calibration data/points is described. In one embodiment, the method includes: (i) selecting a portion (e.g., a percentage) of the given set of calibration points, (ii) using that portion to fit a calibration curve, (iii) applying the calibration curve to the full set of calibration points; (iv) computing mean absolute relative difference (MARD) between the calibration and reference data; (v) repeating steps (i) to (iv) for all combinations of portions of the calibration data; and (vi) selecting the calibration curve that provides the least MARD. In one implementation, approximately 80% of the given set of calibration data is selected in step (i). In another implementation, 70% to 90% of the given set of calibration data is selected in step (i).

In one variant of the method, the calibration mode is performed while the sensing apparatus is implanted in vivo. In another variant of the method, the calibration mode is applied post hoc to previously collected blood analyte data so as to correct it for one or more errors.

In another aspect, a computer readable apparatus is disclosed. In one embodiment, the computer readable apparatus comprises a storage medium (e.g., magnetic, solid state, optical, or other storage medium) having at least one computer program disposed thereon and readable by a computerized apparatus. The at least one computer program includes, in one variant, a plurality of instructions which, when executed on the computerized apparatus, cause operation of one or more blood analyte sensor apparatus in a calibration and/or error correction mode, prior to operating the one or more apparatus in an analyte detection mode.

In yet another aspect of the disclosure, a computerized network apparatus is disclosed. In one embodiment, the network apparatus includes a cloud-based server apparatus configured to store, and optionally analyze, blood analyte data for a population of users (e.g., persons with at least partly implanted blood analyte sensors, and/or their caregivers). In one variant, the network apparatus includes AI (artificial intelligence) or ML (machine learning) algorithms which allow individual user's data to be analyzed (whether in light of their own prior data, and/or in light of data from one or more other users) in order to identify patterns, correlations, or other features or artifacts within the data which may then be leveraged by the user's (or other user's) implanted device to reduce or remove error components or enhance calibration functionality.

In another aspect, implantable blood analyte evaluation apparatus and methods which provide reduced calibration requirements (e.g., reduced number of calibration events per unit time, or reduced total number of calibration events) for a user are disclosed. In one variant, the reduced requirements for e.g., fingersticking or calibration from another source (such as a percutaneous CGM device) are provided via at least one of algorithmic selection of most relevant data, or active causation of the user to provide the most relevant data. In one such variant, one or more algorithms operative to execute on an implanted blood analyte sensor or an external platform associated therewith determine most optimal times or windows of time for the user to provide fingerstick data; i.e., those times or windows when the data will have maximal impact or relevance for calibration curve or error determination. In one implementation, these times or windows are derived based on statistical data considerations (e.g., when the data will provide one or more desired statistical benefits) as opposed to a purely physiologic consideration (e.g., when the user has last ingested a certain type of food, whether the user is waking or sleeping, etc.).

In yet another aspect, methods and apparatus for providing enhanced statistical data selection for use in one or more of error correction or calibration development associated with a blood analyte signal are disclosed. In one variant, historical data within a prescribed period or number of samples is selected based on its efficacy or relevancy for error correction. In one implementation, the data relates to blood glucose level (e.g., pO2 data), and data is selected algorithmically by logic on the implanted sensor (and/or logic on an external platform) based on one or more asymmetric considerations such as hypoglycemia versus hyperglycemia proximity. For instance, enhanced statistical analysis or selection may be performed in cases where proximity and/or trending of blood glucose data to a hypoglycemic event exists, as compared to cases where proximity and/or trending to a hyperglycemic event (the latter which is less potentially deleterious to the user).

In another aspect of the disclosure, methods and apparatus for utilizing sensor data from different sensor locations within a multi-sensor blood analyte sensor array are described. In one embodiment, the sensors are disposed at different locations within a common sensor array that is implanted within a user, and error data relating to a target sensor is adjusted based on weighting of error data from other physically disparate sensors; i.e., each of the disparate sensors Is used to provide “weighted insight” on performance of the target sensor.

In still another aspect of the disclosure, a portable electronic apparatus is disclosed. In one embodiment, the portable electronic apparatus includes a portable receiver device configured to train an implanted blood analyte sensor via, inter alia, wireless data communication therewith.

In another aspect, methods and apparatus for selecting data points for use in calibration in an on-demand fashion are disclosed. In one variant, the on-demand selection is triggered via an algorithmic determination that an implanted sensor is operating within a prescribed or desired operating region or envelope.

In a further aspect, an integrated circuit (IC) apparatus is disclosed. In one embodiment, the IC apparatus includes one or more individual ICs or chips that are configured to contain or implement computerized logic configured to enable one or more of the error detection, correction and/or calibration techniques described herein.

In a further aspect of the disclosure, computer readable apparatus comprising a storage medium is described. In one embodiment, the storage medium has at least one computer program rendered thereon, and the at least one computer program is configured to, when executed by a processor apparatus of a computerized device, cause the computerized device to: algorithmically determine at least one period of time wherein an efficacy or utility of blood analyte sensor calibration data will be below an acceptable level; and cause at least one computerized blood analyte sensor calibration process to adjust obtainment of calibration data for a blood analyte sensor based at least on the algorithmic determination.

In another embodiment, the at least one computer program is configured to, when executed by a processor apparatus of a computerized implantable blood analyte sensing device having a plurality of first sensing elements and a plurality of second sensing elements, cause the computerized implantable blood analyte sensing device to: algorithmically identify an error in a blood analyte concentration measured by a first one of the plurality of first sensor elements at a first one of a plurality of second sensor elements; and based at least in part on the algorithmic identification, identify at least one combination of measurements of a set of the plurality of first sensor elements to estimate the blood analyte concentration at the first one of the plurality of second sensor elements.

In a further aspect of the disclosure, a method for determining a correction for use with blood analyte data generated by an implantable blood analyte sensing device is described. In one embodiment, the method includes: obtaining blood analyte data from the blood analyte sensing device; algorithmically identifying one or more reference data points which meet a prescribed criterion, the prescribed criterion relating to one or more effects on a calibration function; utilizing at least the one or more identified reference data points to algorithmically determine the calibration function; and applying the calibration function to at least a portion of the blood analyte data to correct for one or more errors within the blood analyte data.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1A is a logical flow diagram illustrating an exemplary embodiment of a method of calibrating a blood analyte sensor using “intelligent” calibration reference data gathering according to the present disclosure.

FIG. 1B is a logical flow diagram illustrating another exemplary method of calibrating a blood analyte sensor using intelligent calibration reference data gathering according to the present disclosure.

FIG. 1C illustrates exemplary data including a calibration line fitted over a number of calibration points (in this example, obtained over a 14 day period) using the calibration method described in FIG. 1B.

FIG. 2A is a side cross-sectional view of one exemplary embodiment of a detector element useful with the techniques of the present disclosure.

FIG. 2B is a top elevation view of a one exemplary embodiment of a fully implantable sensor apparatus according to the present disclosure, including an exemplary detector array comprising a plurality of paired differential detector elements of the type shown in FIG. 2A.

FIG. 3A-3C are top elevation views of, respectively, (i) a second exemplary embodiment of a fully implantable sensor apparatus useful with the techniques of the present disclosure, (ii) an exemplary detector array, and (iii) a detector group of the exemplary array.

FIG. 4A is a simplified top elevation view of a sensor area of one embodiment of an implantable sensor apparatus useful with the various aspects of the present disclosure.

FIGS. 4B and 4C are graphs illustrating glucose and oxygen offset bias associated with an exemplary configuration of a differential pair sensor.

FIGS. 4D and 4E illustrate an exemplary embodiment of a calibration curve (sensing accuracy) quality without removal (FIG. 4D) and with removal (FIG. 4E) of the bias associated with a differential pair sensor.

FIG. 5 shows a flow diagram of a method of identifying oxygen concentration error in a differential signal blood analyte sensor and compensating for the error according to aspects of the present disclosure.

FIG. 5A illustrates a method of improving calibration accuracy using a set of given calibration points, according to aspects of the present disclosure.

FIG. 6 illustrates examples of calibration lines fitted over a number of test points selected from a set of calibration points.

FIGS. 6A and 6B illustrate examples of calibration lines fitted over a number of test points selected from a set of calibration points for prior art and inventive approaches, respectively.

All Figures © Copyright 2016-2022 GlySens Incorporated. All rights reserved.

### DETAILED DESCRIPTION

Reference is now made to the drawings, wherein like numerals refer to like parts throughout.

### Overview

In exemplary aspects, the present disclosure provides method and apparatus which improve: (i) accuracy of calibration of a blood analyte sensor, (ii) accuracy of calculated analyte concentration data provided by the blood analyte sensor, and/or (iii) user experience during use of and interaction with the blood analyte sensor.

In one embodiment, the accuracy of an analyte sensor is improved using at least one of: a) “intelligently” selecting calibration/reference points for use during calibration, b) identifying and compensating for systematic error related to e.g., sensor spatial heterogeneity that is inherent in a differential sensor system (such as a glucose/oxygen system), c) using a hybrid bootstrapping/least squares algorithmic process to selectively discard a desired fraction of more erroneous calibration points, or d) using a second continuous or semi-continuous analyte monitoring device (e.g., a wearable, percutaneous glucose monitor) during calibration of an implanted analyte sensor as an opportunistic calibration source.

Accordingly, in some embodiments, user experience can be improved by reducing the number and/or frequency of fingerstick or other calibration data events that a user is required to perform (or by eliminating fingerstick testing altogether) without sacrificing implanted device calibration accuracy.

The blood analyte sensor in some embodiments disclosed herein is a glucose monitor utilizing oxygen-based sensing, and the blood analyte detectors include oxygen and glucose electrodes (e.g., arranged as differential sensor pairs as in the exemplary Model 100 analyte sensor manufactured by the Assignee hereof and described in co-owned and co-pending U.S. patent application Ser. Nos. 13/559,475, 14/982,346, 15/170,571, 15/197,104, 15/359,406, 15/645,913, and 16/233,536 each previously incorporated herein; or arranged as differential sensor groups as in the exemplary GEN 3 Model also manufactured by the Assignee hereof and described in co-owned and co-pending U.S. patent application Ser. Nos. 16/443,684 and 16/453,794, each previously incorporated herein).

In one embodiment, the aforementioned implantable sensor (e.g., an oxygen-based sensor for detection of blood glucose level) is used in conjunction with either a local receiver apparatus (e.g., a wearable local receiver apparatus) in data communication with a parent platform (e.g., a user's mobile device), or a dedicated receiver and processor apparatus. The sensor and/or the receiver apparatus are configured for performing calibration operations after implantation of the sensor. During such calibration, the sensor system collects and calculates time-stamped blood analyte level data (BA_{cal} data), and receives externally generated time-stamped blood analyte level reference data (BA_{ref} data) such as e.g., blood analyte data obtained from fingersticking or blood analyte data obtained from other continuous or semi-continuous sensor devices.

In some configurations, the blood analyte sensor (or an apparatus in communication with the blood analyte sensor) includes logic which can identify opportune times (or time intervals) for obtaining blood analyte level reference data (e.g., using a fingerstick test) and notify the user of the blood analyte sensor, a medical practitioner, and/or another apparatus that can obtain the reference measurements.

In other implementations, blood analyte level reference data useful for calibrating an implanted blood analyte sensor is obtained from a secondary or ancillary “automatic” blood analyte sensor, such as a percutaneous continuous glucose monitoring (CGM) device that is temporarily (e.g., for the duration of a calibration operation) worn by the user.

In yet other implementations, a sensor calibration method is disclosed in which the blood analyte sensor can identify and compensate for systematic error in calculation of the blood analyte level. In one variant, the blood analyte sensor relies on data from several pairs of sensor elements (e.g., glucose and oxygen sensor elements) to calculate a differential blood analyte signal (e.g., based on glucose-oxygen ratios). An algorithm is used to: i) identify an error in the blood oxygen concentration (oxygen partial pressure pO2) measured by an oxygen sensor element at a specific glucose sensor element, and ii) identify a combination of measurements of all oxygen sensors located on the blood analyte sensor to more accurately estimate the blood oxygen concentration at the specific glucose sensor.

In yet other implementations, improved methods of calibrating a blood analyte sensor using a given set of calibration data (i.e., the sensor blood analyte level data and time-matched blood analyte level reference data) are disclosed. In one configuration, the calibration method involves using a hybrid least squares and bootstrapping approach to selectively choose only a portion of available calibration points on which to base a calibration curve, in order to minimize a mean absolute relative difference (MARD) error. Using this approach, outlying/erroneous calibration data points can be selectively culled or disregarded, thereby increasing the accuracy of the calibration and reducing MARD.

In various embodiments, the foregoing methods and associated apparatus related to improving the accuracy of a blood analyte sensor calculations before, during and after calibration can be combined in various ways. For example, the hybrid least squares/bootstrap calibration methodology and logic can be applied to “intelligently” collected calibration data, in effect providing the benefits of both approaches. As another example, the pO2 compensation method can be applied to raw blood analyte sensor data before the sensor data is used as part of a calibration data set. As yet another example, an accurate and “hassle-free” calibration of an implanted sensor device can be performed by using a secondary continuous blood analyte sensor (for inter alia, provision of a great number of reference data points), and by discarding outlier calibration points using the hybrid calibration algorithm.

Additionally, the foregoing sensor training/calibration methods can be repeated (as necessary, on a prescribed schedule, or according to yet another basis) to maintain sensor accuracy throughout the implantation lifetime, even as disease presentation or other physiological or lifestyle characteristics of the user (including foreign body response) change over that same time.

### DETAILED DESCRIPTION OF EXEMPLARY EMBODIMENTS

Exemplary embodiments of the present disclosure are now described in detail. While these embodiments are primarily discussed in the context of a fully implantable glucose sensor, such as those exemplary embodiments described herein, and/or those set forth in U.S. Patent Application Publication No. 2013/0197332 filed Jul. 26, 2012 entitled “Tissue Implantable Sensor With Hermetically Sealed Housing;” U.S. Pat. No. 7,894,870 to Lucisano et al. issued Feb. 22, 2011 and entitled “Hermetic Implantable Sensor;” U.S. Patent Application Publication No. 2011/0137142 to Lucisano et al. published Jun. 9, 2011 and entitled “Hermetic Implantable Sensor;” U.S. Pat. No. 8,763,245 to Lucisano et al. issued Jul. 1, 2014 and entitled “Hermetic Feedthrough Assembly for Ceramic Body;” U.S. Patent Application Publication No. 2014/0309510 to Lucisano et al. published Oct. 16, 2014 and entitled “Hermetic Feedthrough Assembly for Ceramic Body;” U.S. Pat. No. 7,248,912 to Gough et al. issued Jul. 24, 2007 and entitled “Tissue Implantable Sensors for Measurement of Blood Solutes;” and U.S. Pat. No. 7,871,456 to Gough et al. issued Jan. 18, 2011 and entitled “Membranes with Controlled Permeability to Polar and Apolar Molecules in Solution and Methods of Making Same;” and U.S. Patent Application Publication No. 2013/0197332 to Lucisano et al. published Aug. 1, 2013 and entitled “Tissue Implantable Sensor with Hermetically Sealed Housing;” PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as those of U.S. patent application Ser. Nos. 13/559,475, 14/982,346, 15/170,571, and 15/197,104, 15/359,406, 15/368,436, and 15/472,091 previously incorporated herein, it will be recognized by those of ordinary skill that the present disclosure is not so limited. In fact, the various aspects of the disclosure are useful with, inter alia, other types of implantable sensors and/or electronic devices.

Further, while the following embodiments describe specific implementations of e.g., biocompatible oxygen-based multi-sensor element devices for measurement of glucose, having specific configurations, protocols, locations, and orientations for implantation (e.g., proximate the waistline on a human abdomen with the sensor array disposed proximate to fascial tissue; see e.g., U.S. patent application Ser. No. 14/982,346 filed Dec. 29, 2015 and entitled “Implantable Sensor Apparatus and Methods” previously incorporated herein), now U.S. Pat. No. 10,660,550, those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes, and/or theories of operation of glucose sensors, sensors other than glucose sensors, such as e.g., sensors for other analytes such as urea, lactate); (iii) other implantation locations and/or techniques (including without limitation transcutaneous or non-implanted devices as applicable); and/or (iv) devices intended to deliver substances to the body (e.g. implanted drug pumps); and/or other devices (e.g., non-sensors and non-substance delivery devices).

As used herein, the term “analyte” refers without limitation to a substance or chemical species that is of interest in an analytical procedure. In general, the analyte itself may or may not be directly measurable, in cases where it is not, a measurement of the analyte (e.g., glucose) can be derived through measurement of chemical constituents, components, or reaction byproducts associated with the analyte (e.g., hydrogen peroxide, oxygen, free electrons, etc.).

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte.

As used herein, the terms “orient,” “orientation,” and “position” refer, without limitation, to any spatial disposition of a device and/or any of its components relative to another object or being, and in no way connote an absolute frame of reference.

As used herein, the terms “top,” “bottom,” “side,” “up,” “down,” and the like merely connote, without limitation, a relative position or geometry of one component to another, and in no way connote an absolute frame of reference or any required orientation. For example, a “top” portion of a component may actually reside below a “bottom” portion when the component is mounted to another device (e.g., host sensor).

As used herein the term “parent platform” refers without limitation to any device, group of devices, and/or processes with which a client or peer device (including for example the various embodiments of local receiver described here) may logically and/or physically communicate to transfer or exchange data. Examples of parent platforms can include, without limitation, smartphones, tablet computers, laptops, smart watches, personal computers/desktops, servers (local or remote), gateways, dedicated or proprietary analyte receiver devices, medical diagnostic equipment, and even other local receivers acting in a peer-to-peer or dualistic (e.g., master/slave) modality.

As used herein, the term “application” (or “app”) refers generally and without limitation to a unit of executable software that implements a certain functionality or theme. The themes of applications vary broadly across any number of disciplines and functions (such as on-demand content management, e-commerce transactions, brokerage transactions, home entertainment, calculator etc.), and one application may have more than one theme. The unit of executable software generally runs in a predetermined environment; for example, the Java© environment.

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java© (including J2ME, Java Beans, etc.) and the like. Applications as used herein may also include so-called “containerized” applications and their execution and management environments such as VMs (virtual machines) and Docker and Kubernetes.

As used herein, the terms “Internet” and “internet” are used interchangeably to refer to inter-networks including, without limitation, the Internet. Other common examples include but are not limited to: a network of external servers, “cloud” entities (such as memory or storage not local to a device, storage generally accessible at any time via a network connection, or cloud-based or distributed processing or other services), service nodes, access points, controller devices, client devices, etc.

As used herein, the term “memory” includes any type of integrated circuit or other storage device adapted for storing digital data including, without limitation, ROM, PROM, EEPROM, DRAM, SDRAM, (G)DDR/2/3/4/5/6 SDRAM, EDO/FPMS, RLDRAM, SRAM, “flash” memory (e.g., NAND/NOR), 3D memory, stacked memory such as HBM/HBM2, and spin Ram, PSRAM.

As used herein, the terms “microprocessor” and “processor” or “digital processor” are meant generally to include all types of digital processing devices including, without limitation, digital signal processors (DSPs), graphics processors (GPs), reduced instruction set computers (RISC), general-purpose (CISC) processors, microprocessors, gate arrays (e.g., FPGAs), PLDs, state machines, reconfigurable computer fabrics (RCFs), array processors, secure microprocessors, virtual machine processors (VMPs or vCPUs), and application-specific integrated circuits (ASICs). Such digital processors may be contained on a single unitary integrated circuit (IC) die, or distributed across multiple components.

As used herein, the term “network” refers generally to any type of telecommunications or data network including, without limitation, hybrid fiber coax (HFC) networks, satellite networks, telco networks, and data networks (including MANs, WANs, LANs, WLANs, internets, and intranets), cellular networks, as well as so-called “mesh” networks and “IoTs” (Internet(s) of Things). Such networks or portions thereof may utilize any one or more different topologies (e.g., ring, bus, star, loop, etc.), transmission media (e.g., wired/RF cable, RF wireless, millimeter wave, optical, etc.) and/or communications or networking protocols.

As used herein, the term “interface” refers to any signal or data interface with a component or network including, without limitation, those of the FireWire (e.g., FW400, FW800, etc.), USB (e.g., USB 2.0, 3.0. OTG), Ethernet (e.g., 10/100, 10/100/1000 (Gigabit Ethernet), 10-Gig-E, etc.), MoCA, LTE/LTE-A, 5G NR, Wi-Fi (802.11), WiMAX (802.16), Z-wave, PAN (e.g., 802.15)/Zigbee, Bluetooth, Bluetooth Low Energy (BLE) or power line carrier (PLC) families.

As used herein, the term “storage” refers to without limitation computer hard drives, memory, RAID devices or arrays, optical media (e.g., CD-ROMs, Laserdiscs, Blu-Ray, etc.), solid state devices (SSDs), flash drives, cloud-hosted storage, or network attached storage (NAS), or any other devices or media capable of storing data or other information.

As used herein, the term “Wi-Fi” refers to, without limitation and as applicable, any of the variants of IEEE-Std. 802.11 or related standards including 802.11 a/b/g/n/s/v/ac/ax/ba, as well as Wi-Fi Direct (including inter alia, the “Wi-Fi Peer-to-Peer (P2P) Specification”, incorporated herein by reference in its entirety).

As used herein, the term “wireless” means any wireless signal, data, communication, or other interface including without limitation Wi-Fi, Bluetooth, 3G (3GPP/3GPP2), HSDPA/HSUPA, TDMA, CDMA (e.g., IS-95A, WCDMA, etc.), FHSS, DSSS, GSM, PAN/802.15, WiMAX (802.16), 802.20, Zigbee®, Z-wave, narrowband/FDMA, OFDM, PCS/DCS, LTE/LTE-A, 5G NR, analog cellular, CDPD, satellite systems, millimeter wave or microwave systems, acoustic, and infrared (i.e., IrDA).

### Sensor Error

Analyte sensor error is an important concept with respect to various aspects of the present disclosure, and hence some background is provided for context.

Sensor error may be due to a variety of different factors, and can be expressed by the mean absolute relative difference (MARD) between the sensor output and a set of comparison measurements (i.e., a reference measurement), or by the frequency of outliers in the comparison. In one example, the relationship between a measured blood analyte level and a reference blood analyte level (taken at a corresponding point in time) can be expressed by Equation (1) below:

BA_{ref}=BA_{cal}−BAₑᵣᵣₒᵣ−e Eqn. (1)

In Equation (1), “BA_{ref}” is a blood analyte level measured using an external source, “BA_{cal}” is a blood analyte level measured by a calibrated implanted sensor, “BAₑᵣᵣₒᵣ” is systematic error due to unmodeled (and possibly user-specific and/or context-specific) system variables, and “e” is error due to random noise.

The BAₑᵣᵣₒᵣ data can be calculated as the mean absolute relative difference (MARD) between the calibrated analyte sensor output (BA_{cal} data) and the external analyte reference data (BA_{ref} data), as set forth in Eqn. (2) below:

$$MARD = \frac{{\sum }_{1}^{N} \vert {BA}_{cal} - {BA}_{ref} \vert}{N} \qquad Eqn . ( 2 )$$

where N is the number of matched pairs of sensor readings and reference samples.

Alternatively (or in tandem), the BAₑᵣᵣₒᵣ data can be calculated as or by the frequency of outliers in the comparison of the BA_{cal} data and the BA_{ref} data, such as using the frequency of occurrences wherein:

|BA_{cal}−BA_{ref}|>A*BA_{ref} Eqn. (3)

where A is a threshold level that may have a value of, e.g., 0.2 or 0.3, so that outliers are determined to be instances where the sensor output differs from the reference value by more than, respectively, 20% or 30% of the reference value.

Additionally or alternatively, the BAₑᵣᵣₒᵣ data can be calculated utilizing one or more other methods (such as e.g., standard deviation, mean absolute difference, etc.).

### Calibration Utilizing “Intelligently” Collected Reference Measurements

In a first aspect of the disclosure, methods of calibrating an analyte sensor by using intelligently collected calibration points (i.e., points of intersection of sensor measurements and reference measurements taken at approximately the same time) are now described. A calibration curve obtained using such calibration points leads to a more accurate estimate of blood analyte concentration during normal operation of the blood analyte sensor, and thus to a reduced error between reference analyte data and analyte sensor data (reduced MARD). In effect, the exemplary embodiments of the methodology algorithmically determine when calibration data will be optimized for inclusion within the extant data set, and may generate a prompt to the user (and/or selectively utilize existing data already collected) to collect data at a prescribed time or within a prescribed window such that the collected data will have the most effect on improving sensor calibration (and ultimately MARD), so that data collection by the user is minimized; i.e., the user does not collect low-efficacy or low-utility data, and as such advantageously minimizes the volume of data or number of points which they must collect via e.g., fingerstick.

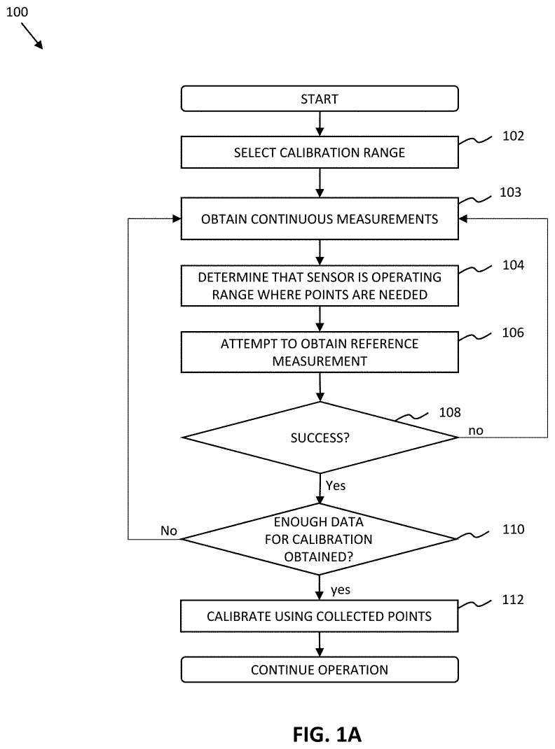

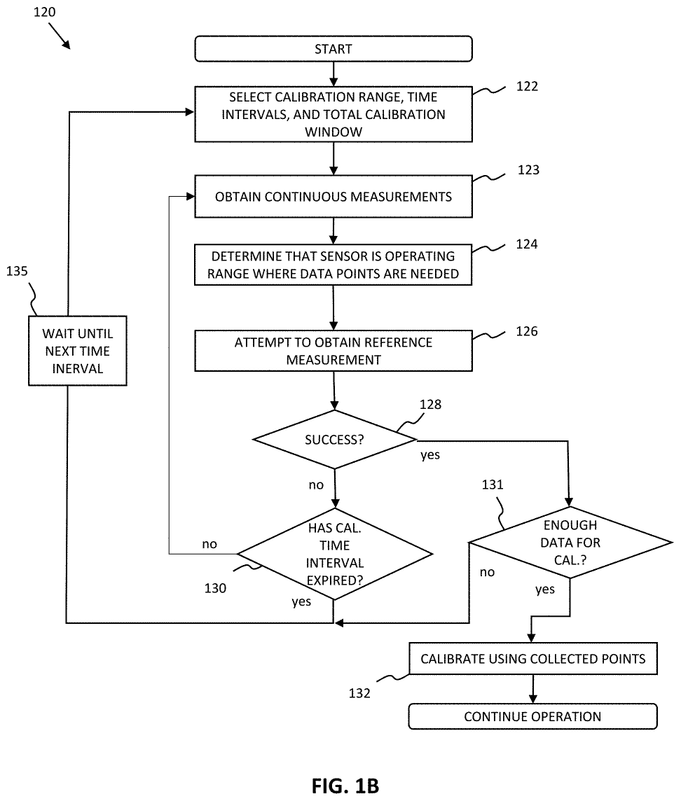

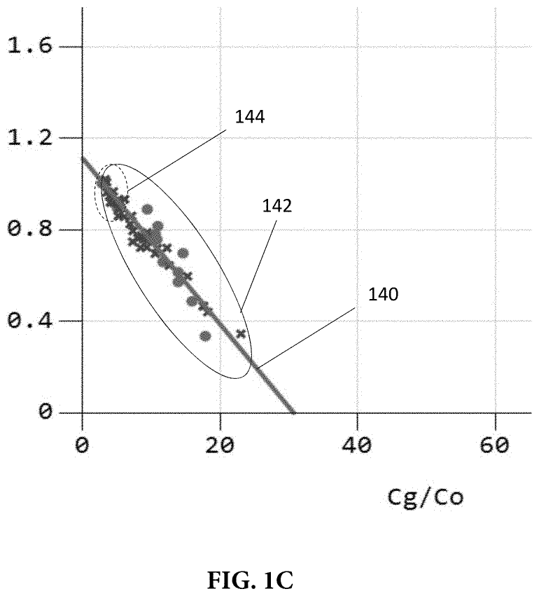

Referring now to FIGS. 1A-1C, exemplary embodiments of the foregoing techniques are described. As discussed elsewhere herein, the implanted analyte sensor may for instance produce a glucose concentration measurement using a glucose-dependent oxygen signal relative to a background oxygen signal. Calibration can be performed using Cg/Co values (glucose to oxygen concentration ratio) as calculated by the sensor and as provided in a reference measurement (e.g., from a fingerstick test).

Within the sensor, the ratio Cg/Co (within an electrode's enzyme-containing membrane) can be defined/measured using another parameter I/I₀, where I is the glucose-modulated current measured at the electrode under a specified condition, and I₀ is the expected current measured under the same specified condition but at zero glucose concentration. As an example, since the oxygen electrode does not respond to the presence of glucose, measured I/I₀ for an oxygen electrode is always expected to be 1 under all conditions. Similarly, in a glucose electrode, the glucose-modulated current decreases with an increase in glucose while other conditions are kept constant (constant pO2, temperature, etc.), leading to lower I/I₀ at higher glucose concentrations. As Cg/Co increases, I/Io should proportionally decrease.

In various embodiments described herein, calibration points are selected over a variety of Cg/Co (or I/Io) values in such a way that: (i) they provide a more accurate calibration curve as compared to regularly collected (i.e., non-specifically evaluated or selected) calibration points, and (ii) fewer calibration points are required to perform a suitably accurate calibration.

FIG. 1A illustrates a flow diagram of one exemplary method 100 of calibrating an implantable oxygen-modulated glucose sensor.

In step 102, a Cg/Co (I/Io) range and point distribution is first selected. As discussed in greater detail below, this selection is performed so that calibration points distributed over the selected range (which are gathered over time, such as over a prescribed historical period) provide a more accurate calibration than calibration points grouped more closely together (over a smaller Cg/Co range). In one embodiment, the range/point distribution may be pre-selected by e.g., a sensor manufacturer, before the analyte sensor is implanted, so that a sensor implantation step may be performed after step 102. In another embodiment, the range may be selected based on measurements of parameters that can only be obtained after the sensor has been implanted, so that step 102 would be preceded by implanting the sensor in the host (such as via the procedures described in U.S. patent application Ser. No. 14/982,346 filed Dec. 29, 2015 previously incorporated herein).

As a brief aside, a then-current calibration line 140 (see FIG. 1C) is algorithmically fitted over the calibration points that have been deliberately chosen to be distributed over a variety of Cg/Co (I/Io) values. Stated simply, selecting data points which have higher variation in Cg/Co (I/Io) values produces a more accurate calibration line than one fitted over the same number of calibration points that are not similarly distributed. This is due in part to the fact that the statistical contribution to the selected calibration function (line) by diverse data points is greater than that from e.g., a similar number of data points which are collocated (i.e., redundant) or nearly along the function line. Greater data diversity produces a more robust calibration result that is expected to perform well over the entire range of the sensor (Cg/Co) than that on a short range covered by the otherwise redundant (collocated) calibration data points.

Furthermore, as an extension of the foregoing concept, calibration points having a more diverse distribution (e.g., over a larger range and not clumped together) may allow for a suitably accurate calibration using fewer data points. This relationship is in some embodiments where e.g., a fingerstick or similar mechanism is used for generating reference measurements, since user experience will be negatively impacted by requiring the user to obtain more reference data than less. As such, the calibration apparatus and methods of the present disclosure can help reduce the number/frequency of fingerstick tests that a user is required to perform while maintaining a suitably high level of calibration accuracy. Conversely, where higher accuracy is required (e.g., a “super-calibration” such as to initially calibrate the functionality of the device after implant or other purposes), a greater number of reference measurements can be made. Hence, the approach described herein can advantageously be scaled (including dynamically, such as via commands from a control algorithm operative on the implant itself, or from external devices such as a user's smartphone or even a percutaneous CGM device) based on desired accuracy, such that either less or more reference measurements can be used in varying scenarios if needed. Under normal operating conditions such as long-term implantation of the sensor, this can translate into many fewer fingersticks or similar measurements that are required over the implantation lifetime of the device, which based on current data may be on the order of two years or more.

In one embodiment, the Cg/Co range or distribution of step 102 can be predetermined/programmed by the sensor manufacturer, such as either a constant value of measured I/Io, time of day, etc. according to a prescribed profile over the anticipated implantation lifetime of the device (e.g., to compensate for long-term physiological or hardware effects such FBR or sensor element efficiency reduction over time). In another embodiment, an appropriate Cg/Co range may be determined by the sensor using various parameters that are measured by or provided (i) internal to the sensor, such as logic which determines operational/state or efficiency, and/or (ii) external to the sensor (e.g., by another device such as a percutaneous CGM, controller algorithm executing on a user device, or even another in vivo or percutaneous device such as a medicant delivery system).

In step 103 of the method 100 of FIG. 1A, the implanted sensor takes continuous electrical current measurements (I/Io) and uses the current measurements to calculate a then-current approximate Cg/Co value.

In step 104, the sensor determines that it is currently operating in a Cg/Co range where one or more calibration points are desired; e.g., the current Cg/Co value calculated by the sensor is (i) within the prescribed calibration range (from step 102), and (ii) not in close proximity to other calibration points that have already be obtained. In one such variant, the extant historical data (e.g., 14 prior data points) are each algorithmically evaluated to determine a “proximity” value relative to a new data point which could/would be used or requested to determine if there is sufficient diversity in the data set, and if so, a signal is generated indicating that reference data collection is requested. As such, the algorithm constantly attempts to build a suitably diverse data set in order to, inter alia, increase accuracy or provide suitable accuracy based on as few data points as possible. In one configuration, a threshold difference or diversity level is used by the algorithm in order to evaluate new (prospective) data; it will be appreciated by those of ordinary skill given the present disclosure however, that other techniques for determining the suitability of a given data point with an extant or planned data set may be used as well consistent with the present disclosure.

In step 106, the sensor attempts to obtain the reference measurement data (assuming it suitably passes the evaluation of step 104) while the sensor remains operating within the desired range. During step 106, the sensor may continue to monitor the Cg/Co levels in order to confirm that it is still operating within the prescribed range as data is collected.

In one embodiment, the sensor either directly or indirectly notifies the user that a reference measurement such as a fingerstick test should be taken, and waits for the user to perform the fingerstick test. In one variant, the fingerstick device can communicate its results to the sensor or to another apparatus in communication with the sensor, such as via wireless data link. In one implementation, this may require the user to connect the fingerstick to the apparatus (e.g., establish a wireless data session or with a cable). In another variant, the user manually enters the results of the fingerstick test (e.g., on a computer, cell phone, etc.) such that they are available to the sensor or to an apparatus in communication with the sensor. Any obtained data (whether via user input or otherwise may also be timestamped, such as upon creation of a data structure or record within the capturing device, or by the sensor upon receipt from an external device) so as to provide a temporal reference for the reference data as well.

In another embodiment, the reference measurement is obtained from another continuous glucose monitoring (CGM) device (e.g., a percutaneous wearable glucose monitor that is temporarily used during the calibration process). In one variant, the sensor instructs the other CGM device, via wireless communication, to take a reference measurement. Alternatively, the CGM device may periodically or otherwise collect reference measurement data, and perform a data push to the implanted device (or respond to a data pull from the implanted device, so as to conserve implanted device battery capacity, such as where the implanted device obviates multiple wireless session establishments and associated processing overhead with a smaller number of pulls or receptions of “bursts” of data from the CGM device). In another variant, the sensor instructs a different apparatus (e.g., an app or program on a cell phone) that is in communication with the other CGM device to request one or more measurements. In yet another variant, the sensor instructs the user to request a measurement from the CGM device (using whichever methods are available).

In one embodiment, if user participation is required in order to obtain the reference measurement, a green light or other visual indicia is displayed on an app/program located on a user device when the implanted sensor is operating in a range where additional calibration points are desired or needed, and no light (or a red light or other indicia) is displayed when the sensor is in a range where additional calibration points are not desired or needed. In other exemplary embodiments, the user can be notified that a reference measurement can be taken via different colors on the app, light/color/sound/haptic vibration change on a dedicated wearable (or implantable) device connected to the sensor, a text message (e.g., sent at the beginning of the window) or another type of electronic message, a push notification to a user's device notification center, etc. In yet another embodiment, the implantable sensor itself may include a haptic device (e.g., powered by the device's battery) to generate a perceptible vibration to alert the user that reference data is required.

In one embodiment, if user participation is required, the sensor can limit the times in which to alert the user that a reference measurement may be taken. In one variant, no notifications (via whatever mechanism) may be sent during certain times of day (e.g., at night when the user is expected to be asleep). In some variants, the times of day may differ depending on the day of the week, the date, etc. In some variants, the user may determine and set the notification rules (e.g., through an app), and update them as needed (e.g., scheduling an important meeting and setting that time as “fenced” or off-limits to notifications from the sensor). In some variants, times that are off-limits to sensor notifications may be dynamically determined by logic on the glucose sensor (directly and/or using other monitoring devices) by making a determination that the user is e.g., asleep, exercising, etc. such as via trends and/or values of blood glucose measurements indicative of such states, and thus it would be inconvenient for the user to take a fingerstick test or perform other types of action.

Referring again to FIG. 1A, in step 108, if a reference measurement is successfully obtained while the sensor is operating within the proper Cg/Co range, the process can move on to step 110. However, if no reference measurement has been obtained and the sensor determines that it has moved out of the appropriate calibration range, the process 100 can return back to monitoring (i.e., repeat steps 103 to 106).

In step 110, the sensor determines whether enough calibration points have been collected to perform an “accurate” calibration (i.e., estimated to produce a prescribed level of accuracy, MARD, or similar metric or greater). In one embodiment, enough calibration points have been collected once a predetermined threshold accuracy is reached (i.e., fit error metric R², MARD, etc. is determined to be low enough). In another embodiment, a number of calibration points to be collected is determined in advance. In one implementation, the predetermined number of calibration points may have an associated requisite characterization associated therewith, such as where the points must have prescribed temporal and/or Cg/Co diversity or meet some other criteria). In yet another embodiment, a combination of threshold accuracy and number of calibration points is used.

In one embodiment, if more (qualifying) calibration points are needed, the method 100 returns to step 103 and iterates. If enough calibration points have been collected, a final calibration curve may be calculated in step 112 using the calibration points collected through the process.

The exemplary calibration process described in FIG. 1A may be configured to provide several improvements to user experience without sacrificing a desired level of accuracy of calibration, including for example via prescribing a time window during which reference measurements should be taken—since a time window in which the reference measurement should be taken may be long (e.g., several minutes up to a few hours), and there is no salient harm in missing the time window (any obtained data may simply be discarded or have its weighting reduced algorithmically depending on proximity to the specified temporal window), the user can take the reference measurement generally at their own convenience. Moreover, under this paradigm, the user needs to take fewer/less frequent reference measurements, which greatly enhances user experience.

Also, in some variants, the calibration algorithm may collect all the data it requires in a shorter time frame (i.e., fewer data points over the same interval produces reduced fingerstick frequency, whereas conversely fewer data points over a reduced interval produces the same frequency but a shorter overall time frame), such that the fully calibrated and working sensor is available for use under the latter model earlier than would otherwise be the case. For instance, in one such variant, a “rapid” calibration protocol or model may be invoked in certain circumstances, such as immediately after implantation of the device, when the user has an impending period of time when they will be unable to obtain reference measurements, such as during a surgical procedure, when they will not have access to a fingerstick or other device, etc.

In yet another embodiment, the reference measurements are obtained opportunistically and directly from an available calibration or reference data source, such as from a secondary continuous analyte sensor, so that i) user action is unnecessary and ii) the calibration process might be performed in a shorter time frame as compared to a scenario where an automated reference data source was not available. For instance, immediately after initial implantation of the sensor device, the user might also apply a percutaneous or other CGM device configured to communicate with the implanted sensor (whether directly or indirectly, such as via an app on the user's smartphone or other device) so as to more rapidly “train” the implanted device initially. As another example, a newly implanted sensor may be trained via an AI/ML-based cloud process configured to obtain sensed data from the implanted sensor (such as via wireless uplink to the user's smartphone and then the cloud process) and based thereon, generate a training regime for execution by the percutaneous CGM (and the implanted device) so as to most efficiently train the latter in the shortest period possible.

Referring to FIG. 1B, another exemplary embodiment of a method 120 of calibrating an implantable oxygen-modulated glucose sensor is described.

In step 122, a Cg/Co range and Cg/Co point distribution is selected, so that calibration points distributed over the range would provide a more accurate calibration than calibration points grouped closer together (e.g., than those distributed over a smaller range). Furthermore, putative calibration time intervals and a total calibration time are also selected. The calibration time intervals are individual, consecutive time windows during which the sensor will attempt to collect a single calibration point. In one exemplary implementation, the calibration Cg/Co range is 0 to 30, the time intervals are 24 hours, and the total calibration time is 14 days.

Steps 123-128 of the calibration method 120 are similar to those in the calibration method 100 described with respect to FIG. 1A. In step 123, the sensor obtains continuous electrical current measurements (I/Io) and uses the current measurements to calculate a then-current approximate Cg/Co value.

In step 124, the sensor determines that it is currently operating in a targeted calibration range (i.e., Cg/Co range where one or more calibration points are desired or needed).

In step 126, the sensor attempts to obtain a reference measurement while the sensor remains within the desired calibration range. In step 128, if a reference measurement is successfully obtained while the sensor is operating within the proper Cg/Co range, the process 120 can move on to step 131, where it is determined whether enough total data for calibration is now available (including the newly collected data from step 126). However, if no reference measurement has been obtained per steps 126 and 128, and the sensor determines that (i) the available window for collecting the reference data has not expired (step 130), and (ii) the sensor has not moved out of the appropriate calibration range, the process proceeds back to step 123 to reattempt acquisition of the new reference point.

It should be noted that if the current time (or time window) expires with no reference measurement taken, the sensor simply continues to operate in the next time period using the existing calibration, and proceeds after an appropriate wait time (e.g., so as to avoid inundating the user with requests for reference data) per step 135, to a new data acquisition at step 122.

Per step 131, the logic of the algorithm 120 determines whether sufficient data is present for a calibration, and if so proceeds to step 132 to calibrate the sensor using the collected data. If sufficient data for calibration has not yet been obtained, the process 120 returns to step 122 to a new data acquisition. In one embodiment of the method 120, the total calibration window (determined at step 122) comprises a moving window; e.g., a moving 14-day period in this example, and the determination of whether sufficient data exists also includes whether the moving window has expired or not. For instance, in one variant of the process 120, a newly implanted device may seek to obtain sufficient reference data by contracting the moving window (or acting irrespective of it), such as by prompting the user or other reference data source to obtain N reference samples (N being a suitable number such that at least an initial calibration can be performed) within a shorter period of time; e.g., over the space of a day or few hours. Thereafter, after the device has been implanted for a period of time, it may implement a longer-term moving window (e.g., 14 days) so that the user/reference source will receive data requests only e.g., once per day. In some implementations, this interval may be relaxed even further, such as after sufficient stability of the device and calibration has been verified (i.e., the user's physiological responses are stable and predictable, and the sensor operation is stable and predictable, as verified by analysis of sensor and reference data obtained during an immediately prior evaluation period of time).

The process of FIG. 1B provides high calibration accuracy with similar improvements to the user experience as that of the calibration process of FIG. 1A, including reducing the frequency/number of reference measurements (e.g., fingerstick tests) that the device needs to obtain, and allowing for variable intervals and total temporal periods of reference data acquisition and sensor calibration.

FIG. 1C illustrates an example of a calibration line fitted over a number of calibration data points obtained over an exemplary 14 day period using the calibration methodology described herein. The “x” data points in the graph indicate calibration data points obtained using non-directed fingerstick measurements once per day (i.e., those taken by the user at effectively random times). The circular dots in the graph indicate calibration points obtained using “intelligent” fingerstick measurements (i.e., taken in time periods as directed by the inventive logic as described herein). The illustrated calibration line 140 has been fitted to the calibration points obtained using intelligent fingerstick measurements. Note that although there are far fewer calibration points obtained via the “intelligent” or directed approach of the present disclosure, the calibration line appears to be a good fit to both sets of data. The non-directed or randomized data obtained include redundant information (e.g., the grouping of points 144 clumped together near the ordinate of the graph of FIG. 1C could be just as well be represented by only a few selected data points), while the intelligently collected calibration points are spread out over a much broader useful range 142, and generally have increased diversity (i.e., variation of data values from the fitted calibration function 140) as compared to the randomly collected data.

It will be appreciated that although the exemplary methods 100 and 120 are described in the context of an oxygen-modulated glucose sensor apparatus, the above principles may be applied to other blood analyte sensors that require calibration using reference measurements, including those for other types of analytes (i.e., non-glucose).

### Exemplary Implantable Sensor

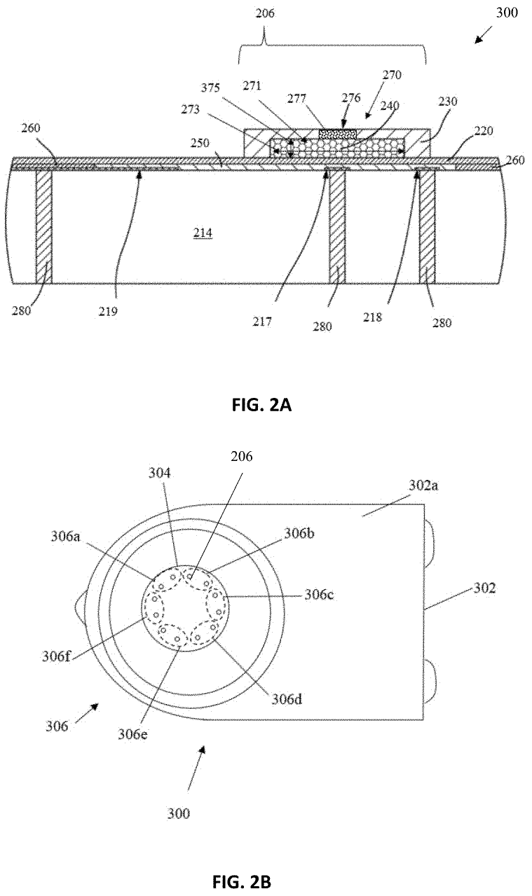

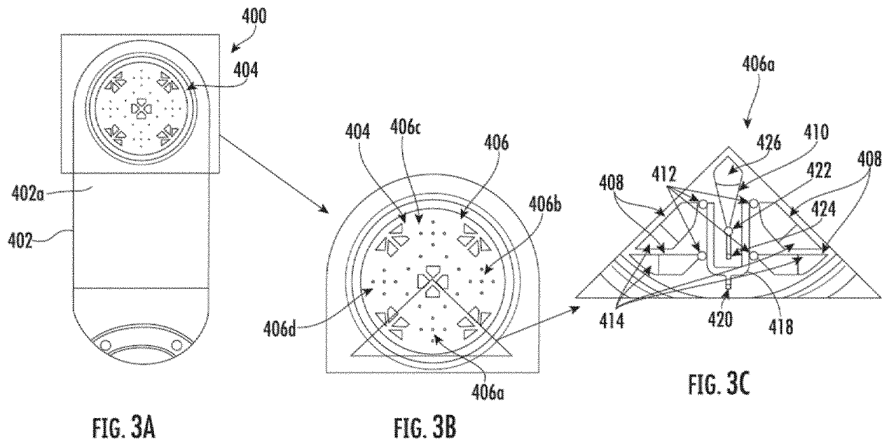

Referring now to FIGS. 2A-3C, exemplary embodiments of an implantable blood analyte sensor useful with the foregoing methodologies are now shown and described.

In one embodiment of the sensor apparatus, as shown in FIG. 2A, an exemplary individual detector element 206 is shown associated with detector substrate 214 (e.g. ceramic substrate), and generally comprises a plurality of membranes and/or layers, including e.g., the insulating layer 260, and electrolyte layer 250, an enzymatic gel matrix 240, an inner membrane 220, an exterior membrane shell 230, and a non-enzymatic membrane 277. Such membranes and layers are associated with the structure of each of the individual detector elements, although certain membrane layers can be disposed in a continuous fashion across the entire detector array surface or portions thereof that include multiple detectors, such as for economies of scale (e.g., when multiple detectors are fabricated simultaneously), or for maintaining consistency between the individual detector elements by virtue of making their constituent components as identical as possible, thereby e.g., minimizing temporal mismatch between paired sensing elements. As shown in FIG. 2A, the detector element 206 further comprises a working electrode 217 in operative contact (by means of the electrolyte layer 250) with a counter electrode 219 and a reference electrode 218, and their associated feedthroughs 280 (details of the exemplary feedthroughs 380 are described in U.S. Pat. No. 8,763,245 to Lucisano et al. entitled “Hermetic feedthrough assembly for ceramic body,” previously incorporated by reference herein). The working electrode 217 comprises an oxygen-detecting catalytic surface producing a glucose-modulated, oxygen-dependent current (discussed infra). A reference electrode 218 comprises an electrochemical potential reference contact to electrolyte layer 250, and a counter electrode 219 is operably connected by means of electrolyte layer 250 to the working electrode 217 and reference electrode 218. An electrical potentiostat circuit (not shown) is coupled to the electrodes 217, 218, and 219 to maintain a fixed potential between the working and reference electrode by passing current between the working and counter electrodes while preferably maintaining the reference electrode at high impedance. Such potentiostat circuitry is known in the art (for an example, see U.S. Pat. No. 4,703,756 to Gough et al. entitled “Complete glucose monitoring system with an implantable, telemetered sensor module,” incorporated herein by reference in its entirety).

In one embodiment, the sensor apparatus utilizes an “oxygen-sensing differential measurement,” by comparison of the glucose-dependent oxygen signal (i.e., from the primary or enzyme-containing sensor elements) to the background oxygen signal (i.e., from the secondary non-enzyme-containing sensor elements) that produces, upon further signal processing, a continuous real-time blood glucose concentration measurement.

In one variant, the enzyme-embedded membrane includes embedded glucose oxidase (GOx) and catalase enzymes and the sensor elements are configured for detection of glucose based on the following two-step chemical reaction catalyzed by GOx and catalase as described in Armour et al. (Diabetes 39, 1519-1526 (1990)):

glucose+O₂→gluconic acid+H₂O₂

H₂O₂→½O₂+H₂O

resulting in the overall enzyme reaction (when catalase is present):

glucose+½O₂→gluconic acid

In one specific implementation of the analyte-modulated detector element, the two enzyme types (GOx and catalase, each in an excess concentration) are immobilized within a gel matrix that is crosslinked for mechanical and chemical stability, and is in operative contact with the working electrode, which is configured to electrochemically sense oxygen. Glucose and ambient oxygen diffuse into the gel matrix and encounter the enzymes, the above reactions occur, and oxygen that is not consumed in the process is detected by the electrode. In embodiments based on “oxygen-sensing differential measurement” (i.e., comparison of an active detector element reading to a background (reference) detector element reading), after comparison of the active oxygen concentration reading with the background oxygen concentration reading, the difference is related to glucose concentration. Thus, hydrogen peroxide produced in the initial GOx catalyzed reaction is digested to oxygen and water via the subsequent catalase catalyzed reaction, and glucose concentration may be determined via detection of oxygen.

In an exemplary embodiment, the enzymatic material 240 comprises a crosslinked gel of hydrophilic material including enzymes (e.g., glucose oxidase and catalase) immobilized within the gel matrix, including a buffer agent and small quantities of a chemical crosslinking agent. The hydrophilic material 240 is permeable to both a large molecule component (e.g. glucose) and a small molecule component (e.g. oxygen). In various embodiments, specific materials useful for preparing the enzymatic material 240, include, in addition to an enzyme component, polyacrylamide gels, glutaraldehyde-crosslinked collagen or albumin, polyhydroxy ethylmethacrylate and its derivatives, and other hydrophilic polymers and copolymers, in combination with the desired enzyme or enzymes. The enzymatic material 240 can similarly be constructed by crosslinking glucose oxidase or other enzymes with chemical crosslinking reagents, without incorporating additional polymers.

The enzymatic material 240 is in operative contact with the working electrode 217 through the inner membrane 220 and the electrolyte layer 250 to allow for the electrochemical detection of oxygen at the working electrode 217 modulated by the two-step chemical reaction catalyzed by glucose oxidase and catalase discussed above. To that end, as glucose and ambient oxygen diffuse into the enzymatic material 240 from the outer (non-enzymatic) membrane 277, they encounter the resident enzymes (glucose oxidase and catalase) and react therewith; the oxygen that is not consumed in the reaction(s) diffuses through the inner membrane 220 and is detected at the working electrode 217 to yield a glucose-dependent oxygen signal. A similarly configured (excluding enzyme) background sensing element produces no reaction with diffused glucose, thereby resulting a glucose-independent oxygen signal.

A hydrophobic material is utilized for inner membrane 220, which is shown in FIG. 2A as being disposed over the electrolyte layer 250. The hydrophobic material is impermeable to the larger or less soluble molecule component (e.g. glucose) but permeable to the smaller or more soluble molecule component (e.g. oxygen). The inner membrane 220 can also be a continuous layer across the entire detector array surface, and thus be a single common layer utilized by all detectors in the detector array (assuming a multi-detector array is utilized). It is noted that the inner membrane 220, inter alia, protects the working electrode 217, reference electrode 218 and counter electrode 219 from drift in sensitivity due to contact with certain confounding phenomena (e.g. electrode “poisoning”), but the working electrode 217 will nonetheless be arranged sufficiently close to the enzymatic material to enable detection of oxygen levels therein.

The (hydrophobic) outer membrane shell 230 is disposed over at least a portion of the enzymatic material 240 (forming a cavity 271 within which the material 240 is contained), and is further configured to include an aperture within a “spout” region 270. It is contemplated that the inner membrane 220 and the membrane shell 230 can be coextensive and therefore be disposed as one continuous membrane layer in which outer membrane shell 230 and inner membrane 220 are of the same uniform thickness of membrane across the individual detector and array, although it will be appreciated that other thicknesses and configurations may be used as well, including configurations wherein the membrane shell 230 is separately provided and adhesively bonded to the inner membrane 220.

As depicted in FIG. 2A, the single spout region 270 of the (primary) detector element 206 forms a small opening or aperture 276 through the membrane shell 230 to constrain the available surface area of hydrophilic enzymatic material 240 exposed for diffusionally accepting the solute of interest (e.g. glucose) from solution. Alternatively, it is contemplated that one or more spout regions (and or apertures within a spout region) can exist per detector element.

The shape and dimension of spout region 270 aids in controlling the rate of entry of the solute of interest (e.g. glucose) into enzymatic material 240, and thus impacts the effective operational permeability ratio of the enzymatic material 240. Such permeability ratio can be expressed as the maximum detectable ratio of glucose to oxygen concentration of an enzymatic glucose sensor, where such a sensor is based on the detection of oxygen unconsumed by the enzyme reaction, and after taking into account the effects of external mass transfer conditions and the enzyme reaction stoichiometry. Detailed discussions of the relationship between membrane permeability ratio and the maximum detectable ratio of glucose to oxygen concentration of oxygen-detecting, enzymatic, membrane-based sensors are provided in “Model of a Two-Substrate Enzyme Electrode for Glucose,” J. K. Leypoldt and D. A. Gough, Analytical Chemistry, 56, 2896 (1984) and “Diffusion and the Limiting Substrate in Two-Substrate Immobilized Enzyme Systems,” J. K. Leypoldt and D. A. Gough, Biotechnology and Bioengineering, XXIV, 2705 (1982), incorporated herein by reference. The membranes of the exemplary detector element described herein are characterized by a permeability ratio of oxygen to glucose of about 200 to about 1 in units of (mg/dl glucose) per (mmHg oxygen). Note that while this measure of permeability ratio utilizes units of a glucose concentration to an oxygen concentration, it is nevertheless a measure of the ratio of oxygen to glucose permeability of the membrane.

As can be seen in FIG. 2B, the exemplary implantable sensor apparatus 300 when viewed from above includes a body 302 having a sensing region 304 disposed on a top surface 302a thereof. A plurality of sensing element pairs 306 (comprised of individual sensors 206 of the type shown in FIG. 2A) are radially arranged and substantially evenly spaced apart within the sensing region 304. An analyte-modulated sensing element and a background sensing element are adjacent pairs of elements such that the arrangement will allow each analyte-modulated element in the pair to remain within the same relatively homogenous region (relative to its paired background element) of the otherwise heterogeneous tissue in which a sensor apparatus 300 is implanted.

It will be appreciated that the background or reference detector element (for each of the differential pairs 306) can have a substantially similar configuration to the analyte-modulated detector element 206. However, different from the analyte-modulated detector element, the background element excludes enzyme from the membrane or material disposed within the cavity (thereby making the element non-responsive to and/or affected by the presence of analyte).

Turning now to FIGS. 3A-3C, in another exemplary embodiment, the sensor apparatus 400 comprises a housing 402 having a sensing region 404 disposed on a top surface 402a thereof. The sensing region 404 includes a plurality of grouped differential detector elements 406 (e.g., four groups of elements). In the illustrated embodiment of the sensor apparatus 400, the signal received from an analyte-modulated electrode is utilized to determine a ratiometric or differential signal relative to a plurality of background electrodes (two or more background electrodes) in order to determine a blood analyte concentration. Such a configuration for a glucose sensor advantageously reduces error in common-mode (background oxygen) signals due to the dispersed spatial arrangement of the background sensing elements relative to the glucose-modulated sensing element, and thereby increases overall accuracy of the sensor. The foregoing sensor element configurations are further disclosed in co-owned U.S. patent application Ser. No. 16/443,684 filed Jun. 17, 2019 and Ser. No. 16/453,794 filed Jun. 26, 2019, each previously incorporated by reference herein.

Specifically, as can be seen in the detailed view shown in FIG. 3C, the exemplary group of sensing elements 406a includes multiple background sensing elements 408 (e.g., four background oxygen elements) associated with and proximate to a single analyte-modulated sensing element 410 (e.g., one glucose-modulated oxygen element). In alternate embodiments, the sensor face may in include additional or fewer groups of sensors, and/or additional or fewer background (oxygen) elements associated with each analyte-modulated (glucose) element. Additionally, in the embodiment shown in FIGS. 3A-3C, each of the sensor element groups has a configuration which is substantially similar to other sensor groups; however, in alternate embodiments, the sensor elements within each group may have a different configuration/arrangement than that of the other groups (e.g., group 406b having a different configuration than group 406a).

Also shown in FIG. 3C, the four background oxygen elements each include a background oxygen (BO) working electrode 412 associated with a BO counter electrode 414. The BO counter electrodes 414 are substantially disposed at opposing lateral sides (proximate to an outer perimeter) of the sensing element group 406. The orientation of the BO counter electrodes toward the outer perimeter of the sensing element group enables a closer arrangement of the BO working electrodes to the glucose sensing element. Specifically, the BO working electrodes 412 are evenly-spaced and arranged around the glucose-modulated (GM) working electrode 422 in a substantially square-shaped configuration, thereby enabling measurement of background oxygen generally within the same microenvironment as the GM electrode. Each of the BO working electrodes is disposed on a U-shaped filament 418, which is configured for association of each of the BO working electrodes 412 to a single (shared) BO reference electrode 420. The BO reference electrode 420 is proximate to the outer perimeter of the sensor group 406a and an outer perimeter of the sensor face. In alternate embodiments, each of the BO working electrodes may be associated with a separate BO reference electrode; however, utilization of a shared BO reference electrode advantageously enables a reduced size of the sensor face.

Also shown in FIG. 3C, the GM sensing element 410 comprises the aforementioned GM working electrode 422, a GM reference electrode 424, and a GM counter electrode 426. The GM electrode 410 is linearly arranged, where the GM counter electrode 426 is disposed proximate to a center of the sensor face, the GM reference electrode 426 is disposed proximate to the BO reference electrode 420, and the GM working electrode 422 is disposed therebetween (i.e., between the GM counter and reference electrodes). In the present embodiment, the GM working electrode 422 and reference electrode 424 are disposed between the arms of the U-shaped filament 418, while the GM counter electrode 426 is outside of the filament. Similar to the orientation of the BO counter electrodes, such arrangement of the GM counter electrode enables a “closer” spatial arrangement or proximity of the GM working electrode to the BO working electrodes (with e.g., an approximate distance of 68 mils therebetween in one particular implementation, although this value may be varied in other implementations).

Calibration with Systematic pO2 Error Correction

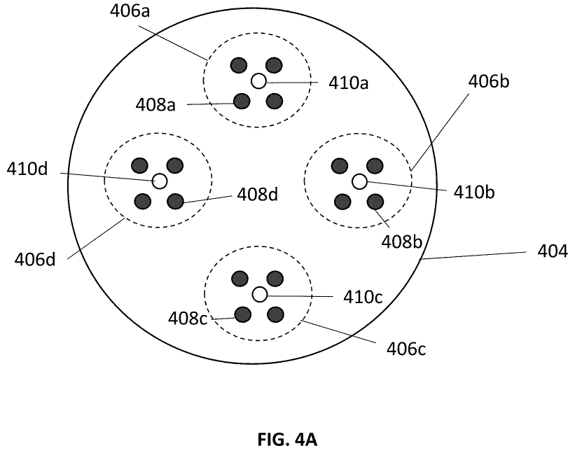

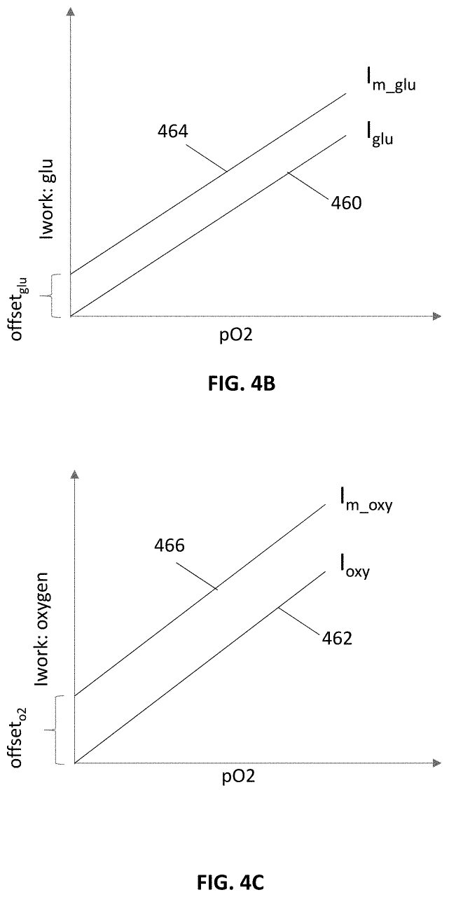

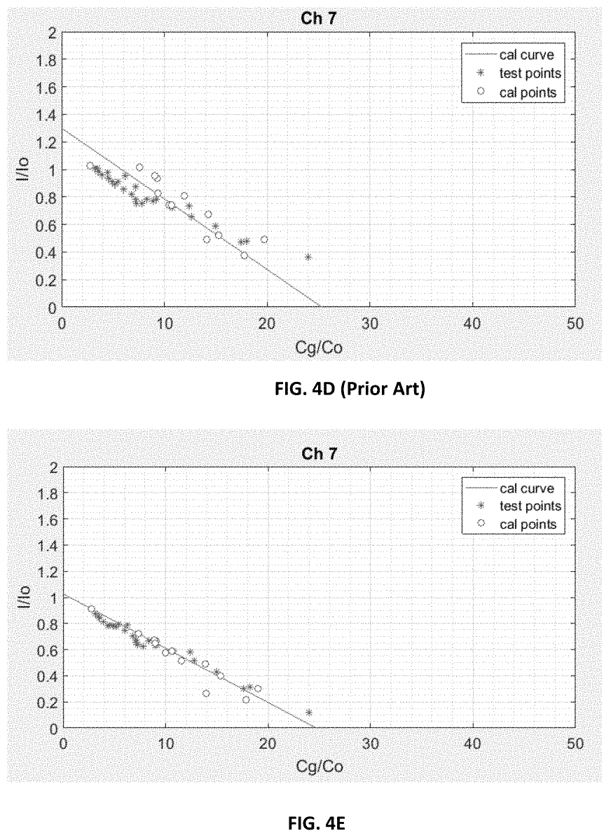

FIG. 4A illustrates a simplified sensor region 404 (as shown in FIG. 3B discussed supra) having four sensing elements (406a-406d), each with an analyte-modulated sensing element (410a-410d) and sets of background sensing elements (408a-408d). In one embodiment, the analyte-modulated sensing elements are glucose sensing elements and the background sensing elements are background oxygen elements. Each set of background sensing elements 408a-d may include four (4) active electrodes surrounding a single active electrode of the analyte sensing element, as described above with respect to FIG. 3C and shown in FIG. 4A, although the illustrated configuration and spatial relationships are merely illustrative of the broader principles.

Although much of the error due to the dispersed spatial arrangement of the background sensing elements relative to the glucose-modulated sensing element is mitigated with the sensor element arrangement of FIG. 3C, the error cannot be completely eliminated using geometry alone. Notably, a minimum physical distance between the glucose and oxygen working electrodes is needed to ensure that the sensing elements continue to operate as glucose and oxygen sensors, respectively; placement too close to one another will in effect cross-contaminate the operation of the other. Since the background sensing element and glucose-modulated sensing element (electrodes) cannot physically be placed at exactly the same location, the electrodes of the background (oxygen) sensing element sets 408 of a sensing element 406 necessarily measure pO2 that is slightly different than the true pO2 at the glucose sensing element 410 of that sensing element 406. In other words, if the glucose sensing element 410a of sensor element 406a calculates glucose based on the pO2 measurement provided to it by its associated oxygen sensing element electrodes 408a, it is using a reference pO2 number that is slightly off from the true/ideal oxygen concentration at the glucose sensing element 410a. This spatial heterogeneity is expressed per Eqn. (4):

Spatial Heterogeneity=reference pO2 sensor pO2. Eqn. (4)

For the purpose of calibration, error can be correlated with a variety of variables (e.g., measured pO2, temperature, spatial heterogeneity, etc.). The pO2 error can be defined per Eqn. (5):

Error=ideal pO2−measured pO2. Eqn. (5)

FIGS. 4B and 4C illustrate bias associated with a differential pair sensor (i.e., a glucose sensor and its oxygen sensor, respectively) seeing slightly different oxygen partial pressures. The bias may be associated with sensor hardware itself, and/or one or more physiological conditions within the subject, but is generally not predictable or controllable for purposes of this discussion.

Assuming that both sensors of the differential pair see the same pO2, and the sensor currents have zero current at zero pO2 (0 mmHg), the currents for the glucose working electrode (FIG. 4B) and the background oxygen working electrode (FIG. 4C) should follow the lines indicated by I_{glu} 460 and I_{oxy} 462, respectively. The ratio of currents I/Io can be expressed per Eqn. (6):

I/Io=B*Cg/Co+A Eqn. (6)

wherein B and A are constants.

However, the sensor operates in non-ideal conditions where the foregoing assumptions are not preserved, and the two sensors of the differential pair do not see the same pO2. The fixed offset in pO2 around the two differential sensors or sensor currents at 0 mmHg leads to error. FIG. 4B illustrates the measured current at the glucose working electrode (I_{m_glu}) 464 having an offset (offset_{glu}). FIG. 4C illustrates the measured current at the background oxygen working electrode (I_{m_oxy}) 466 having an offset (offset_{O2}). The ratio of the currents can be expressed per Eq. (7):

(I−x)Io=B*Cg/Co+A Eqn. (7)

where x accounts for bias (offset) in both the glucose and oxygen electrodes.

FIGS. 4D and 4E illustrate the calibration curve (sensing accuracy) quality with and without removing the bias associated with a differential pair sensor (i.e., a glucose sensor and its oxygen sensor, respectively) seeing slightly different oxygen partial pressures. FIG. 4D shows higher scatter around the calibration curve due to the unresolved (unaccounted) bias in prior art that may be associated with sensor hardware itself, and/or one or more physiological conditions within the subject, but is generally not predictable or controllable for purposes of this discussion. FIG. 4E shows reduced scatter around the calibration curve due to the bias correction as noted in Eqn. (7).

With the foregoing as background, exemplary methodologies for addressing the foregoing errors during operation are now described.

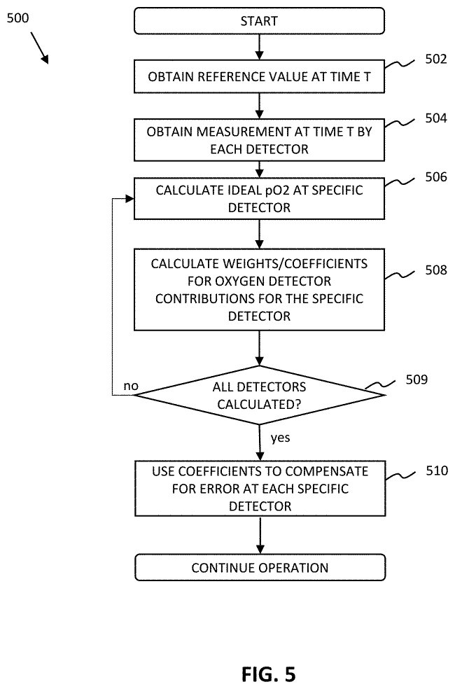

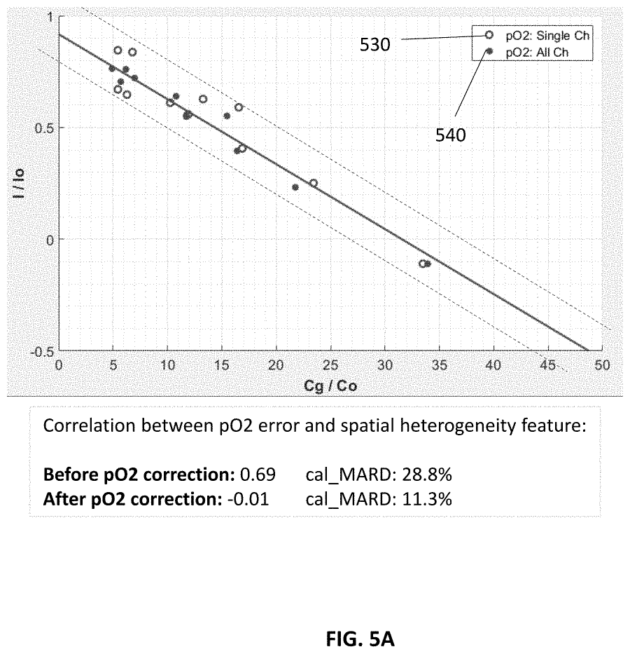

FIG. 5 shows a flow diagram of one embodiment of a method 500 of identifying pO2 error and performing pO2 correction for individual sensing elements 406 of an analyte sensor (i.e., compensating for the fixed offset errors present in each differential sensor pair). Advantageously, the method 500 can be implemented with an analyte sensor as shown in FIGS. 3A-3C and 4A, or with another type of analyte sensor that similarly uses differential measurements to calculate blood analyte concentration (whether glucose or otherwise).

In step 502 of the method 500, at least one reference value is obtained from an external source at a known time(s) (e.g., the reference measurements can be time-stamped fingerstick measurements, or data derived from another source such as a percutaneous CGM device).

In step 504, the measured glucose and oxygen concentrations of each detector element 406 are obtained. In one embodiment, the glucose concentration measured by each glucose sensing element 410, and pO2 measured by each individual background sensing element 408 at the known time(s), are obtained. It will be appreciated that in configurations such as that of FIGS. 3C and 4A where multiple background electrodes are used (e.g., 4), the values of each may be considered in the aggregate, or even individually if desired.

Additionally, the measurements of steps 502 and 504 may be obtained in real time, or collected from a set of historical data that has been stored for later analysis, or even combinations of the foregoing.

In step 506, the measurements collected in steps 502 and 504 are used to calculate the ideal pO2 at a specific glucose detector 410 of a specific sensing element 406 (i.e., what is the pO2 that an oxygen detector should have measured at the glucose sensor). For example, ideal (true) pO2 may be calculated for the glucose sensing element 410a of the element 406a (see FIG. 4A) using the methods described in co-owned U.S. patent application Ser. No. 16/233,536, previously incorporated by reference herein, although it will be appreciated that other techniques may be used consistent with the present disclosure.

In step 508 of the method 500, linear regression is applied to the pO2 values measured by all four background detector sets (408a, 408b, 408c, 408d) to obtain the ideal pO2 value at the first glucose sensing element 410a. Using step 508, the ideal pO2 value at glucose sensing element 410a may be obtained using a combination of different weights/contributions of the four background detector sets (or even individual elements of each set, for a greater degree of granularity). This can be calculated using the Eqn. (8):

Ideal pO2(N)=x1(N)*pO2(1)+x2(N)*pO2(2)+x3(N)*pO2(3)+x4(N)*pO2(4)+x5(N) Eqn. (8)

where:
x1(N)=the weight/contribution of the first background detector set 410a to the calculation of oxygen concentration at the Nth sensor;
x2(N)=the weight of the second background detector set 408b to the Nth sensor;
x3(N)=the weight of the third background detector set 408c to the Nth sensor;
x4(N)=the weight of the fourth background detector set 408d to the Nth sensor;
x5(N)=the fixed pO2 offset observed at the Nth sensor; and
Ideal pO2(N)=the determined true/ideal oxygen partial pressure at the Nth glucose sensor 410.

For example, in one exemplary embodiment, the contributions of the four different sensor measurements to the ideal oxygen concentration of the first glucose sensor 410a can be determined by calculating the coefficients x1(1), x2(1), x3(1), x4(1) and x5(1) in Eqn. (9):

Ideal pO2(1)=x1(1)*pO2(1)+x2(1)*pO2(2)+x3(1)*pO2(3)+x4(1)*pO2(4)+x5(1). Eqn. (9)

Note that the above equation applies to a sensor having four sensing elements 406a, 406b, 406c, 406d, and thus four coefficients/weights (x) and an offset. However, a similar calculation can be applied to a sensor having more or fewer than four sensing elements 406, and further the individual number of weights can be correlated to (and calculated for) the actual number of oxygen background detector electrodes (4 in this example also) for increased granularity if desired. The level of granularity of the analysis may also be dynamically varied during operation if desired, such as based on criteria such as measured stability of glucose concentration, state of the user (e.g., ambulatory or asleep), age of the implant (e.g., new, or implanted for a period of time), and/or yet other factors.

Returning to FIG. 5, in step 509, the method 500 determines whether the pO2 calculation has been performed for every sensing element 406a, 406b, 406c, 406d of the analyte sensor. In other words, steps 506-508 should be individually and separately applied to each of the sensing elements. If the calculation has not been applied to each of the sensing elements, the process repeats steps 506-508 for the next sensing element until the sensor has been fully characterized.

In step 510, once the coefficients or weights have been identified for each sensing element relative to a given “target” element, they can be utilized to estimate the oxygen concentration at each of the glucose sensing elements 410a, 410b, 410c, 410d.

For example, the identified coefficients (x1(1), x2(1), x3(1), x4(1), x5(1)) of the first sensor 406a, in conjunction with the pO2 values measured by the four background detector sets 408a, 408b, 408c, 408d, can be used to more closely estimate the actual pO2 value at the first glucose sensor element 410a (estimated pO2(1)) using Eqn. (10):

estimated pO2(1)=x1(1)*pO2(1)+x2(1)*pO2(2)+x3(1)*pO2(3)+x4(1)*pO2(4)+x5(1). Eqn. (10)

Thus, when calculating the glucose concentration from the first sensor element 406a, the background oxygen partial pressure is provided by the identified weighted mix/combination of background oxygen sensor sets 408a, 408b, 408c, 408c (as determined in step 508), and not merely by the background oxygen sensor set 408a that is associated with the first sensor 406a (i.e., is closest to the first sensor).

Note that the concentration of oxygen at the first glucose sensor 410a may be heavily weighted to the contribution from the first background oxygen sensor 408a (e.g., x1(1) is closer to 1.0 than other values). However, at least some of the other sensors provide non-zero contributions to the calculation (i.e., at least one of x2(1), x3(1), and x4(1) is non-zero).

Similarly, the identified coefficients (x1(2), x2(2), x3(2), x4(2), x5(2)) of the second sensor 406b, in conjunction with the pO2 values provided by the four background detector sets 408a, 408b, 408c, 408d, are used to estimate pO2 at the second glucose sensor element 410b using Eqn. (11):

estimated pO2(2)=x1(2)*pO2(1)+x2(2)*pO2(2)+x3(2)*pO2(3)+x4(2)*pO2(4)+x5(2). Eqn. (11)

Similar calculations are applied to the rest of the glucose sensor elements 410c, 410d.

As such, the methodology 500 advantageously makes use of data from other disparately located sensor elements 406, the latter providing some (weighted) input or insight into the glucose measurement overall. This approach of estimating oxygen partial pressure at each of the glucose sensor elements in effect compensates for some of the systematic error associated with the analyte sensor and the specific physical conditions affecting different areas of the sensor (e.g., the particular vascularization or lack thereof around each of the sensor elements 406). Since this systematic error is relatively stable, the coefficients calculated once in step(s) 508 may be used thereafter during regular operation of the glucose sensor (step 510). More broadly, the inventors hereof recognize that the systematic pO2 error in individual sensor elements of a blood analyte sensor may be highly specific to the conditions around and inside individual portions of the sensor and only determinable in vivo (i.e., after implantation of the sensor). On the other hand, once calculated, the pO2 error remains stable over time. Thus, it can be successfully accounted/corrected for after implantation using e.g., the context-specific algorithm described in the present disclosure, thereby providing significantly improved accuracy in terms of, e.g., mean absolute relative difference (MARD) between the sensor output and a comparison or calibrated measurement, or by the frequency of outliers in such comparisons or calibrations, as compared to conventional implantable blood analyte sensor systems.

The graph of FIG. 5A shows one embodiment of a correlation between pO2 error and spatial heterogeneity. Before performing a pO2 correction (e.g., using the method 500 of FIG. 5), the differential signal I/Io is calculated using measurements provided to each glucose sensor element by a single background oxygen sensor/set (single channel), producing the uncorrected or single-channel data set 530 (circles on the graph). After performing a pO2 correction, each glucose sensor element obtains a background oxygen measurement from a weighted combination of all background oxygen sensors (all channels), producing the corrected data set 540 (dots on the graph). As can be seen in FIG. 5A, the pO2 correction leads to reduced systematic scatter of data around the calibration line, and reduced error (MARD). The effect is particularly pronounced at lower values of Cg/Co, as small absolute error in reference pO2 (and subsequently I/o or Cg/Co) corresponds to large percentage error at low Cg/Co. Thus, even if the calibration curve does not move, the accuracy of the differentially calculated glucose concentration is improved.

### Calibration Enhancement Using Selective Algorithmic Data Elimination

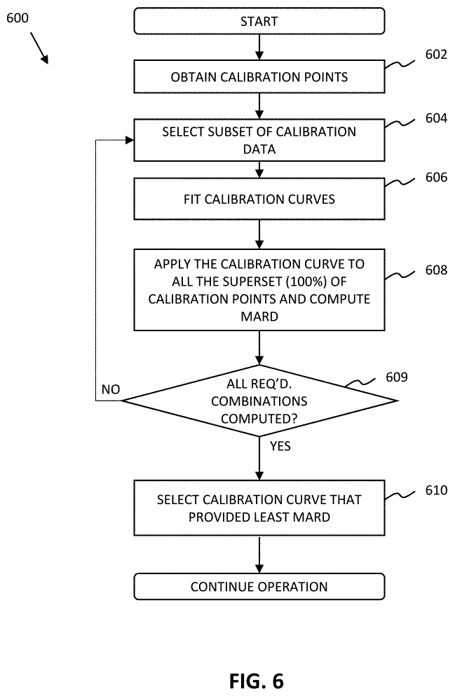

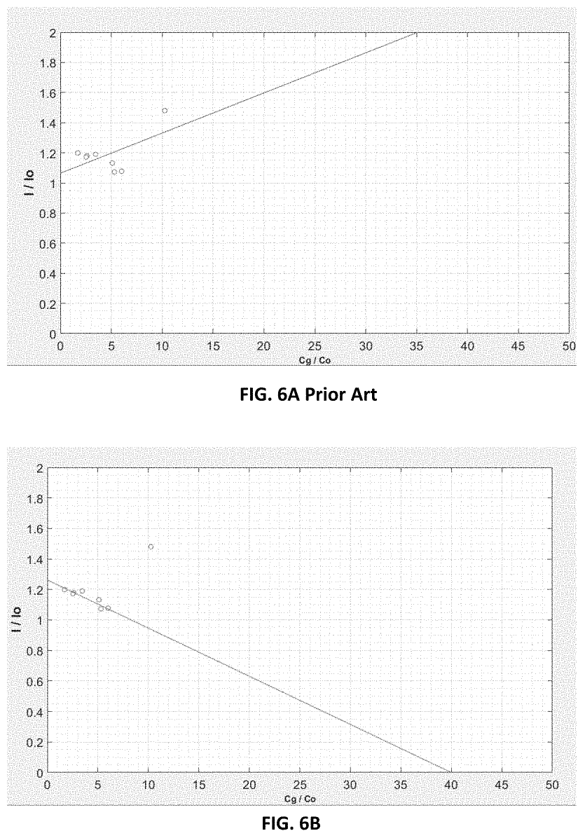

Referring now to FIGS. 6-6B, exemplary methods for enhancing sensor calibration accuracy using selective data elimination techniques are shown and described in detail. At a high level, these exemplary techniques mathematically focus on one or more subsets of all available calibration data (i.e., “bootstrap”: subset(s) with desired size or other properties) in order to determine a calibration function or curve, and then apply (test) the developed function or curve back onto the complete data set. In one particular implementation of the algorithm, a hybridized least-squares and bootstrapping approach is utilized, as discussed in greater detail below. This approach advantageously reduces the computational overhead or burden associated with prior art iterative approaches while also providing a high level of calibration accuracy. Specifically, under a typical prior art boostrapping regime, a final of global solution is converged upon using numerous (e.g., hundreds) of iterations of statistical calculations (e.g., for a or mean), in effect identifying a “mean of means.” For instance, a subset of data points is e.g., randomly selected, desired statistical parameters evaluated, and then the process iterated with a different randomly selected subset, until a solution is converged on to a desired confidence level (e.g., 95% CI).

However, in the present context, two important considerations are recognized: (i) for MARD, there is no “global minima” or globally unique solution, and (ii) the exemplary ICGM apparatus does not have the luxury of performing the computationally intensive process described above, due to inter alia, time, processing power, and battery (electrical) power considerations. Accordingly, the exemplary method described herein seek to both reduce the foregoing computational burden on the sensor processing logic (and/or its proxy process), as well as rapidly converging on a suitably accurate calibration curve or function and lowest MARD.

FIG. 6 illustrates one embodiment of a method of improving calibration efficiency and accuracy using a subset of given calibration points. In step 602, a plurality of calibration points/data is obtained. In one embodiment, the calibration points include a set of time-stamped Cg/Co (or I/Io) measurements taken by an ICGM, and a matching set of self-monitored blood glucose (SMBD) values; e.g., Cg/Co values with the same or similar (temporally correlated) time stamps. The SMBG calibration data—i.e., reference analyte data—can be obtained for example from fingerstick reference measurements and/or secondary sensor measurements. In one implementation, the calibration data is reference data provided by a user from 14 fingerstick measurements. In another implementation, the calibration data includes reference data collected by a non-implanted (e.g., percutaneous) CGM.

In practice, portions of the reference data (e.g., a single fingerstick measurement) may be obtained by the implanted sensor logic (or its proxy, such as an external user device, dedicated receiver, or even cloud process), the associated time stamp or other temporal reference determined, and the implanted sensor measurement identified or taken shortly thereafter, and the process subsequently repeated for the remaining reference data points as they are captured. Alternatively, the reference measurements and the implanted sensor measurements may be captured effectively in parallel with one another, and once the set of reference measurements to be used have been identified (whether all or a subset of those obtained by or provided to the implanted sensor or proxy), then temporally correlated implanted sensor (ICGM) measurements for those selected reference data points can be identified, such as from a larger pool of measured data which has been stored.

Returning to FIG. 6, in step 604, a portion or subset of the calibration or reference data is selected (whether before or after identification of the temporally correlated ICGM data). In one embodiment, 80% of the calibration points are selected. In other embodiments, more or fewer (e.g., 90% or 60%) of the calibration points are selected. In one implementation, 12 fingerstick measurements out of 14 are selected by the algorithm.

It will be appreciated that various criteria for reference data point subset selection or filtering may be used in accordance with the method of FIG. 6. For instance, a constant or static assumption as to the percentage of data points may be used, along with a random selection algorithm. Hence, in one embodiment, every time the method returns to step 604 (from step 609), the algorithm selects a different portion or subset of the total calibration points according to the same prescribed selection criteria. However, the present disclosure also contemplates that the subset selection criteria may (i) be dynamic on an inter-iteration basis (e.g., 12 points on first iteration, less or more on a subsequent iteration, and so forth), and/or (ii) be based on some characteristic of each data point (e.g., such as based on type/source, confidence level of the data, etc.). For example, the algorithm may in some implementations be configured to preferentially select as many of the X (e.g., 12) points of a static threshold derived from a first source (e.g., percutaneous CGM) as possible, and any remainder from fingerstick-sourced data, or vice versa. Or, as another example, the algorithm may select the subset (12) of the 14 data points based on values which have a largest aggregated mean temporal diversity (i.e., which display the highest time difference between each other data point as possible). As another example, the data may be selected such that maximal Cg/Co value diversity exists. Some reference data likewise may have a higher propensity to be a “data outlier” by virtue of its value, collection, and/or relationship to other data. Numerous other examples of “intelligent” subset selection that may be used consistent with the present disclosure will be appreciated by those of ordinary skill.

In step 606, a calibration curve is fitted only to the selected portion of the calibration points, such as via a least-squares algorithm or similar approach.

In step 608, the calibration curve calculated in step 606 is extrapolated or applied to all (100%) of the available calibration points (e.g., 14 in the foregoing example), and error/MARD for that particular calibration curve is calculated and stored.

In one embodiment, at step 609, if MARD has been computed for all possible combinations of calibration data (e.g., every combination of 12 calibration points out of 14), or otherwise a termination criterion has been met (e.g., 50% of the permutations have been calculated), the method proceeds to step 610. If MARD has not been computed for all combinations (or the termination criterion has not been met), the method returns to step 604 and selects a different subset of calibration data. In some variants, the termination criterion (e.g., predetermined number of repetitions or percentage of possibilities) may be made dependent on the total number of calibration points available and/or selected for the subset. For example, in one such implementation, if 14 reference data points are available, and 12 are selected for the subset, 50% of all possible combinations for the selected 12 points may be sufficient to characterize the data set, whereas if 10 data points are selected for each subset, 75% of all possible combinations of the selected 10 points may be required for suitable characterization.

In step 610 of the method, the calibration curve that meets the desired criterion (e.g., provides the smallest MARD) is then selected for subsequent use during operation.

Hence, on each “pass” of the example calculation (the number of which may also be statically or dynamically selected), 12 different randomly selected points of the 14 total are selected, statistically analyzed and a curve fitted to the 12-point data set (e.g., using a least squares approach). The curve is then extrapolated or bootstrapped onto the larger (e.g., 14 point) data set, and MARD calculated (i.e., between the reference data and developed calibration function). After the prescribed number of iterations are completed, the curve/function which produces the best (here, lowest) MARD value is selected for use in calibrating the ICGM data (whether historical or newly obtained thereafter).

It will be appreciated that the method 600 of fitting a calibration line to all available data points based on the MARD calculated from the calibration line fitted to only a subset of points allows the calibration algorithm to effectively discard a percentage of calibration points (e.g. 20% of points where 80% is used as the selection criterion) that are determined to contribute less to, or even detract from, more accurate calibration.

Moreover, this method 600 of evaluating a given set of calibration points may be combined with any combination of methods discussed previously in the present disclosure in order to further improve the calibration accuracy of an analyte sensor. It can be selectively applied e.g., based on user context such as ambulatory or sleeping, point within a term of implantation, under individual sensor element failure conditions due to e.g., FBR over time, availability of external reference data sources, the source of the reference data, and/or yet other factors.

FIGS. 6A and 6B illustrate examples of calibration lines fitted over a number of test points selected from a set of calibration points.

It will be recognized that while certain embodiments of the present disclosure are described in terms of a specific sequence of steps of a method, these descriptions are only illustrative of the broader methods described herein, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure and claimed herein.

While the above detailed description has shown, described, and pointed out novel features as applied to various embodiments, it will be understood that various omissions, substitutions, and changes in the form and details of the device or process illustrated may be made by those skilled in the art without departing from principles described herein. The foregoing description is of the best mode presently contemplated. This description is in no way meant to be limiting, but rather should be taken as illustrative of the general principles described herein. The scope of the disclosure should be determined with reference to the claims.

What is claimed is:

1. Computer readable apparatus comprising a storage medium, the storage medium having at least one computer program rendered thereon, the at least one computer program configured to, when executed by a processor apparatus of a computerized device, cause the computerized device to:
algorithmically determine at least one period of time wherein an efficacy or utility of blood analyte sensor calibration data will be below an acceptable level; and
cause at least one computerized blood analyte sensor calibration process to adjust obtainment of calibration data for a blood analyte sensor based at least on the algorithmic determination.

2. The computer readable apparatus of claim 1, wherein the adjustment of the obtainment of calibration data for the blood analyte sensor based at least on the algorithmic determination comprises causing obtainment of the calibration data so as to at least partly avoid the at least one time period.

3. The computer readable apparatus of claim 1, wherein the computerized device comprises an implantable blood analyte sensor apparatus comprising the blood analyte sensor.

4. The computer readable apparatus of claim 1, wherein the computerized device comprises a non-implantable wireless enabled device in wireless data communication with an implantable blood analyte sensor apparatus comprising the blood analyte sensor.

5. The computer readable apparatus of claim 1, wherein the acceptable level comprises a prescribed threshold, the prescribed threshold determined dynamically via algorithmic analysis of one or more error sources associated with the blood analyte sensor.

6. The computer readable apparatus of claim 1, wherein the at least one computer program is further configured to, when executed by the processor apparatus of the computerized device, cause the computerized device to:
based at least on the algorithmic determination, cause at least one computerized blood analyte sensor calibration process to adjust obtainment of blood analyte measurement data to be within a prescribed window of time relative to the obtainment of the calibration data.

7. The computer readable apparatus of claim 1, wherein the algorithmic determination of the at least one period of time wherein an efficacy or utility of blood analyte sensor calibration data will be below an acceptable level comprises identification of one or more systematic errors related to a spatial heterogeneity of the blood analyte sensor.

8. The computer readable apparatus of claim 1, wherein the adjustment of the obtainment of calibration data for a blood analyte sensor based at least on the algorithmic determination comprises use of an algorithmic process to selectively discard a desired fraction of more calibration data points determined to have a prescribed level of error associated therewith.

9. The computer readable apparatus of claim 8, wherein the prescribed level of error relates to one or more systematic errors related to a spatial heterogeneity of the blood analyte sensor.

10. The computer readable apparatus of claim 8, wherein the use of an algorithmic process to selectively discard a desired fraction of more calibration data points comprises use of a hybrid bootstrapping/least squares algorithmic process.

11. The computer readable apparatus of claim 1, wherein the at least one computer program is further configured to, when executed by the processor apparatus of the computerized device, cause the computerized device to establish data communication with an analyte monitoring device different than the blood analyte sensor during calibration of the blood analyte sensor to obtain blood analyte measurement data for use as an opportunistic calibration source.

12. Computer readable apparatus comprising a storage medium, the storage medium having at least one computer program rendered thereon, the at least one computer program configured to, when executed by a processor apparatus of a computerized implantable blood analyte sensing device having a plurality of first sensing elements and a plurality of second sensing elements, cause the computerized implantable blood analyte sensing device to:
algorithmically identify an error in a blood analyte concentration measured by a first one of the plurality of first sensor elements at a first one of a plurality of second sensor elements; and
based at least in part on the algorithmic identification, identify at least one combination of measurements of a set of the plurality of first sensor elements to estimate the blood analyte concentration at the first one of the plurality of second sensor elements.

13. The computer readable apparatus of claim 12, wherein:
the plurality of first sensor elements comprises a plurality of blood oxygen sensor elements;
the plurality of second sensor elements comprises a plurality of blood glucose sensor elements; and
the blood analyte concentration measured by a first one of the plurality of first sensor elements comprises an oxygen partial pressure (pO2) measurement.

14. The computer readable apparatus of claim 13, wherein:
the plurality of blood oxygen sensor elements and the plurality of blood glucose sensor elements are configured in a plurality of pairs, each of the pairs comprising a blood oxygen sensor element and a blood glucose sensor element; and
the implantable blood analyte sensing device includes computerized logic configured to utilize signals from a plurality of the pairs to calculate a differential blood analyte signal.

15. The computer readable apparatus of claim 14, wherein the utilization of signals from a plurality of the pairs to calculate a differential blood analyte signal comprises calculation of the differential blood analyte signal based at least in part on one or more ratios relating glucose signals to oxygen signals.

16. The computer readable apparatus of claim 12, wherein the algorithmic identification of an error in a blood analyte concentration measured by a first one of the plurality of first sensor elements at a first one of a plurality of second sensor elements comprises algorithmic identification of at least one of (i) a systematic error due to one or more unmodeled system variables, or (ii) an error due to random noise.

17. The computer readable apparatus of claim 16, wherein:
the at least one of: (i) a systematic error due to one or more unmodeled system variables, or (ii) an error due to random noise, comprises the one or more unmodeled system variables; and
the systematic error due to the one or more unmodeled system variables comprises systematic error due to one or more unmodeled system variables which are at least one of a) user-specific or b) context-specific.

18. The computer readable apparatus of claim 16, wherein:
the at least one of: (i) a systematic error due to one or more unmodeled system variables, or (ii) an error due to random noise, comprises the one or more unmodeled system variables; and
the systematic error due to the one or more unmodeled system variables comprises systematic error calculated as a mean absolute relative difference (MARD) between a calibrated analyte sensor output and external analyte reference data according to:
$$MARD = \frac{{\sum }_{1}^{N} \vert {BA}_{cal} - {BA}_{ref} \vert}{N}$$
where N is a number of matched pairs of sensor readings and reference data samples.

19. A method for determining a correction for use with blood analyte data generated by an implantable blood analyte sensing device, the method comprising:
obtaining blood analyte data from the blood analyte sensing device;
algorithmically identifying one or more reference data points which meet a prescribed criterion, the prescribed criterion relating to one or more effects on a calibration function;
utilizing at least the one or more identified reference data points to algorithmically determine the calibration function; and
applying the calibration function to at least a portion of the blood analyte data to correct for one or more errors within the blood analyte data.

20. The method of claim 19, wherein:
the blood analyte sensing device comprises an oxygen-based differential blood glucose sensor; and
the algorithmically identifying the utilizing, and the applying are each performed while the blood analyte sensing device is in vivo by one or more computer programs resident to execute on a digital processor apparatus of the blood analyte sensing device.
